(19) United States
(12) Patent Application Publication
(10) Pub. No.: US 2023/0292718 A1
(43) Pub. Date: Sep. 21, 2023

(54) METHOD FOR CONSTRUCTING MODEL OF VITILIGO AND THE USE OF THE MODEL

(71) Applicant: NATIONAL INSTITUTE OF BIOLOGICAL SCIENCES, BEIJING, Beijing (CN)

(72) Inventors: Ting CHEN, Beijing (CN); Zijian XU, Beijing (CN)

(21) Appl. No.: 17/922,535

(22) PCT Filed: Apr. 30, 2020
(86) PCT No.: PCT/CN2020/088055

§ 371 Date: Oct. 31, 2022

(51) Int. Cl. A01K 67/027; C12N 5/071; C12Q 1/6883

(52) U.S. Cl. CPC A01K 67/0271; C12N 5/0625; C12Q 1/6883; A01K 2207/10; A01K 2207/12; A01K 2227/105; A01K 2267/0325; C12Q 2600/158; C12Q 2600/112; C12Q 2600/106; C12Q 2600/118

(57) ABSTRACT

Provided is a drug target for vitiligo. The drug target is IFN-γ signaling. It also provides a method for establishing vitiligo animal model, or a vitiligo induction method in an animal, and the established vitiligo animal model. Further, provided is a method for screening candidate drugs for treating vitiligo using the said animal model.

### INTRODUCTION

Vitiligo is a chronic condition that causes white patches developed on the skin, in which pigment cells (melanocytes) are lost. Vitiligo affects 0.5-1% of the population, and occurs in all races. In 50% of sufferers, pigment loss begins before the age of 20, and in about 80% it starts before the age of 30 years. In 20% of sufferers, other family members also have vitiligo. Males and females are equally affected.

Vitiligo is thought to be a systemic autoimmune disorder, associated with deregulated innate immune response, although this has been disputed for segmental vitiligo. There are many drugs for treating vitiligo in the prior art, and the mechanisms that the treatment is based on are different, but the treatment effects are not ideal. The reasons come down to unclear mechanisms.

Previously developed vitiligo mouse model relies on adoptive transfer of melanocyte-specific CD8+ T cells isolated from PMEL-specific TCR transgenic mice into Krt14-Kit1 transgenic mice (ref). The requirement of 2 transgenic alleles and a transfer procedure to induce vitiligo in mouse skin limits additional genetic alterations that can be efficiently introduced to carry out in-depth functional studies in vivo. Other existing vitiligo mouse models either wait for spontaneous vitiligo development on transgenic mice with slow progression and low efficiency (ref); or utilize ectopically expressed melanocyte antigens that require complicated virus packaging system or specialized gene gun delivery equipment deterring general application. Most importantly these vitiligo mouse models all rely on artificial stimulations that do not occur in patients.

Establishing ideal animal model of vitiligo and investigating definite mechanisms or exact pathogenesis of vitiligo are needed so as to screen candidate drugs for treating vitiligo.

### SUMMARY OF THE INVENTION

The inventors of the present invention used immunofluorescent staining to analyze skin of vitiligo patients at the border region of depigmented lesion and pigmented perilesion, and found the depigmented lesion region contains slightly more CD45+ immune cells than the perilesion region, but majority of the infiltrated immune cells are concentrated at the junction area between the lesion region and perilesion region. This intriguing distribution pattern of CD45+ immune cells indicates certain recruitment mechanism is orchestrating the local aggregation of immune cells, which drives the expansion of depigmented region in patient skin.

The inventors of the present invention used single cell RNA-seq to analyze all cell types present in patient skin in order to test what skin resident cells are involved in mediating immune cells recruitment, distinguish different disease states and reveal major associated signaling pathways. The result showed that progressive state vitiligo skin contains more CD8+ cytotoxic T cells that express significant amount of IFNG compare to quiescent state patients and healthy donors. Corresponding melanocytes in progressive state vitiligo skin up regulate genes involved in immune response, especially response to IFN-γ.

The inventors of the present invention used immunofluorescent staining to examine the spatial distribution pattern of CD8+ T cells and IFN-γ responsive cells in patient skin. To quantify this spatial correlation, the vitiligo skin was divided into three regions based on skin pigmentation and T cell infiltration: depigmented lesion region, T cell infiltrated region (TIR) and adjacent pigmented perilesion region. Quantification shows the CD8+ T cell density to be in TIR is significantly higher than those in both lesion and perilesion regions. It was found that the pSTAT1 (Phosphorylated Signal Transducer and Activator of Transcription 1)+IFN-γ responsive cell density is significantly higher in TIR compared to lesion region and perilesion region. Importantly, the density of CD8+ T cells positively correlates with the density of pSTAT1+ cells. This result showed that the regional response to T cell secreted IFN-γ correlates with progressive disease state.

The inventors of the present invention developed a new vitiligo mouse model through inoculating mice with melanoma cells, and receiving immunotherapy using antibody for treating melanoma. The vitiligo mouse model revealed that response to T cell secreted IFN-γ is required for local CD8+ T cell aggregation and cytotoxic activity in skin.

Together graft and vitiligo induction experiments, it is demonstrated that skin resident IFN-γ responsive cells are required for local CD8+ T cell recruitment and activation in skin. In particular skin dermal cells are capable of mediating this effect.

Next, the fibroblast mosaic knockdown experiments not only validate the result that IFN-γ responsive dermal fibroblast is the main cell type mediating local CD8+ T cell aggregation and activation, they also further reveal the IFNGR1-JAK1-STAT1 signaling axis in fibroblasts is required for mediating CD8+ T cell local aggregation and activation. Most importantly these experiments show that in a field with uneven fibroblast response to IFN-γ, T cells preferentially aggregate towards regions with high IFNGR1-JAK1-STAT1 signaling.

The inventors of the present invention further validated that IFN-γ responsive fibroblasts are sufficient to mediate local CD8+ T cells aggregation in vivo and in vitro through secreted chemokines such as CXCL9, CXCL10 and CCL19.

The inventors of the present invention also showed that intrinsic IFN-γ response differences of anatomically distinct human fibroblasts correlate with regional disease variations.

In the first place, the present invention provides a drug target for vitiligo. The drug target is IFN-γ signaling. Preferably, the drug target is the IFN-γ signaling within cells in skin. The cells in skin may be endothelial cells, dermal cells, smooth muscle cells or immune cells in skin. Preferably, the drug target is IFN-γ signaling within dermal fibroblast of skin.

The present invention demonstrates that certain IFN-γ responsive cell(s), or response to IFN-γ is essential for CD8+ T cell local aggregation and cytotoxic activity in skin using IFNGR1 KO (IFN-γ receptor 1 knock-out) induced vitiligo mouse. Specifically, the present invention demonstrates that IFN-γ responsive skin dermal fibroblast is the main cell type mediating local CD8+ T cell aggregation and activation. That is to say, fibroblast is the main cell type responsible for orchestrating local CD8+ T cell aggregation and activation after being stimulated by IFN-γ in autoimmune skin. In particular, IFN-γ responsive fibroblasts alone are sufficient to orchestrate local CD8+ T cells aggregation.

Skin dermal fibroblasts are necessary and sufficient to induce CD8+ T cell local aggregation and activation in response to IFN-γ in vitiligo skin. IFN-γ responsive fibroblasts mediate CD8+ T cells aggregation through secreted factors. Therefore, preferably, the drug target is fibroblast-specific secreting chemokines induced by IFN-γ signaling. IFN-γ signaling induced fibroblast-specific secreted chemokines control regional T cell recruitment. Preferably, the chemokines may be CCL5, CCL8, CCL19, CXCL3, CXCL9 and/or CXCL10. IFN-γ responsive fibroblasts are sufficient to mediate CD8+ T cells aggregation in vivo and in vitro through secreted chemokines such as CXCL9, CXCL10 and CCL19.

T cells preferentially aggregate towards regions with high IFNGR1-JAK1-STAT1 signaling. Preferably, IFNGR1-JAK1-STAT1 signaling axis in fibroblasts is required for mediating CD8+ T cell local aggregation and activation. Therefore, preferably, the drug target is IFNGR1-JAK1-STAT1 signaling within dermal fibroblast of skin.

Fibroblasts from anatomically distinct body positions show intrinsic differences in IFN-γ response. The intrinsic differences of anatomically distinct human dermal fibroblast correlate with the vitiligo incidence at different body positions. The present invention shows large variations of vitiligo incidence in the eight body regions: with hand back, chest and back skin regions to be the most susceptible to vitiligo, while palm and arm skin to be the least susceptible. Vitiligo incidence positively correlates with the intrinsic IFN-γ response of skin fibroblast. Therefore, fibroblasts from anatomically distinct body positions can be used as different drug targets in IFN-γ response. In particular, fibroblasts from hand back, chest and back skin can be used as drug targets in IFN-γ response.

The chemokine genes CCL2, CXCL3, CXCL9, CXCL10, and CXCL11 are mainly upregulated in the skin fibroblasts from hand back and foot back. QPCR validations confirmed the intrinsic IFN-γ response differences of fibroblasts from different anatomic positions. The correlation of IFN-γ response enrichment score to vitiligo incidence at different body positions was evaluated and the result shows vitiligo incidence positive correlates with the intrinsic IFN-γ response of skin fibroblast. Thus, the chemokine genes CCL2, CXCL3, CXCL9, CXCL10, and CXCL11 upregulated in the skin fibroblasts from hand back and foot back could be used as the drug targets.

In the second place, the present invention provides a method for establishing vitiligo animal model, or a vitiligo induction method in an animal, comprising inoculating the animal with melanoma cells, injecting CD4 depletion antibody and removing the melanoma.

The melanoma cell may be B16F10 or B16 melanoma cell.

Preferably, the tumors are surgically removed before expanding and metastasizing.

Preferably, the animal may be mouse, rat, canine, pig or cat.

As for a mouse, the tumors are surgically removed after the volume of the melanoma reaching 62.5-256 mm³ in order to prevent tumor cells expanding and metastasizing.

Preferably, 9-week-old C57 mice are inoculated with B16F10 melanoma cells in the right flank of dorsal skin; then CD4 depletion antibody is injected on Days 4 and 10. The tumors are surgically removed on Day 12 to prevent tumor cells expanding and metastasizing.

Because melanocytes in mouse dorsal skin are located in hair follicles but not in epidermis, so mouse tail skin is used for vitiligo analysis since it contains epidermis localized melanocytes similar to human skin. In tail skin, epidermis depigmentation becomes visually apparent at 16 weeks post induction, mainly depending on the natural turnover rate of pigmented keratinocytes on skin surface.

Compared to control mice tail skins that contain almost no CD8+ T cells, at Day40 after vitiligo induction, vast number of CD8+ T cells infiltrated into tail skin epidermis, accompanied by significant loss of melanocytes.

After vitiligo induction, CD8 depletion antibody is used and results in complete block of CD8+ T cell infiltration in tail skin epidermis and rescue of melanocytes loss. So the vitiligo induction method efficiently triggers endogenous activated CD8+ T cells infiltrating skin that results in loss of native melanocytes located in epidermis similar to autoimmune vitiligo patients.

The main experimental advantage of our vitiligo induction method or the method for establishing vitiligo mouse model is that it only utilizes commercially available reagents and can efficiently induce patient like vitiligo pathologies on any mice lines, even with genetic alterations such as knockout, conditional knockout, or transgene; hence will enable to ask in-depth mechanistic questions and screen effective drugs.

In the third place, the present invention provides a vitiligo animal model induced through inoculating the animal with melanoma cells, injecting CD4 depletion antibody and removing the melanoma.

The melanoma cell may be B16F10 or B16 melanoma cell. Preferably, the tumors are surgically removed before expanding and metastasizing.

Preferably, the animal is mouse, rat, canine, pig or cat.

As for a mouse, the tumors are surgically removed after the volume of the melanoma reaching 62.5-256 mm³ in order to prevent tumor cells expanding and metastasizing.

Preferably, 9-week-old C57 mice are inoculated with B16F10 melanoma cells in the right flank of dorsal skin; then CD4 depletion antibody is injected on Days 4 and 10. The tumors are surgically removed on Day 12 to prevent tumor cells expanding and metastasizing.

Because melanocytes in mouse dorsal skin are located in hair follicles but not in epidermis, so mouse tail skin is used for vitiligo analysis since it contains epidermis localized melanocytes similar to human skin. In tail skin, epidermis depigmentation becomes visually apparent at 16 weeks post induction, mainly depending on the natural turnover rate of pigmented keratinocytes on skin surface.

Compared to control mice tail skins that contain almost no CD8+ T cells, at Day40 after vitiligo induction, vast number of CD8+ T cells infiltrated into tail skin epidermis, accompanied by significant loss of melanocytes.

After vitiligo induction, CD8 depletion antibody is used and results in complete block of CD8+ T cell infiltration in tail skin epidermis and rescue of melanocytes loss. So the vitiligo induction method efficiently triggers endogenous activated CD8+ T cells infiltrating skin that results in loss of native melanocytes located in epidermis similar to autoimmune vitiligo patients.

Preferably, the present invention provides an IFNGR1-JAK1-STAT1 overexpressed transgenic animal.

Previously developed vitiligo mouse model relies on adoptive transfer of melanocyte-specific CD8+ T cells isolated from PMEL-specific TCR transgenic mice into Krt14-Kit1 transgenic mice (ref). The requirement of 2 transgenic alleles and a transfer procedure to induce vitiligo in mouse skin limits additional genetic alterations that can be efficiently introduced to carry out in-depth functional studies in vivo. Other existing vitiligo mouse models either wait for spontaneous vitiligo development on transgenic mice with slow progression and low efficiency; or utilize ectopically expressed melanocyte antigens that require complicated virus packaging system or specialized gene gun delivery equipment deterring general application. Most importantly these vitiligo mouse models all rely on artificial stimulations that do not occur in patients. The hallmark of human vitiligo disease, which is epidermal melanocyte loss, has not been carefully reported.

In the fourth place, the present invention provides a skin of an animal, wherein the melanocytes in the skin is reduced.

Preferably, the skin is a skin showing depigmentation. Preferably, the skin is the tail skin or back skin of an animal. Preferably, the skin is hand back, foot back, chest, leg or arm skin of an animal.

Preferably, the animal is mouse, rat, canine, pig or cat.

Preferably, the skin is obtained from induced through inoculating the animal with melanoma cells, injecting CD4 depletion antibody and removing the melanoma.

The melanoma cell may be B16F10 melanoma cell.

Preferably, the tumors are surgically removed before expanding and metastasizing.

Preferably, the animal is mouse, rat, canine, pig or cat.

As for a mouse, the tumors are surgically removed after the volume of the melanoma reaching 62.5-256 mm³ in order to prevent tumor cells expanding and metastasizing.

The volume of the melanoma is calculated based on the formula: 0.5×a×b×b, wherein a is the length of the long axis of the tumor, and b is the length of the short axis of the tumor.

Preferably, 9-week-old C57 mice are inoculated with B16F10 melanoma cells in the right flank of dorsal skin; then CD4 depletion antibody is injected on Days 4 and 10. The tumors are surgically removed on Day 12 to prevent tumor cells expanding and metastasizing.

Because melanocytes in mouse dorsal skin are located in hair follicles but not in epidermis, so mouse tail skin is used for vitiligo analysis since it contains epidermis localized melanocytes similar to human skin. In tail skin, epidermis depigmentation becomes visually apparent at 16 weeks post induction, mainly depending on the natural turnover rate of pigmented keratinocytes on skin surface.

In the fifth place, the present invention provides an isolated cell of skin, wherein the cell is IFN-γ responsive.

The cell includes but is not limited to keratinocyte, melanocyte, fibroblast, endothelium cell, smooth muscle cell, and dendritic cell. Preferably, the cell is fibroblast. Preferably, the cell is dermal fibroblast. Preferably, the cell is dermal fibroblast of mouse tail skin.

Skin resident IFN-γ responsive cells are required for local CD8+ T cell recruitment and activation in skin. In particular, skin dermal fibroblast is the main cell type mediating local CD8+ T cell aggregation and activation. IFN-γ responsive fibroblasts are sufficient to mediate CD8+T cells aggregation in vivo and in vitro through secreted chemokines such as CXCL9, CXCL10 and CCL19. Therefore, IFN-γ responsive fibroblasts from dermis may be used as cells for investigating mechanisms of vitiligo and/or for screening candidate drugs of vitiligo.

IFNGR1-JAK1-STAT1 signaling in fibroblasts is required for mediating CD8+ T cell local aggregation and activation. Injection of IFNGR1 KO fibroblasts into the tail skin of IFNGR1 KO mice did not result in local CD8 T cell aggregation after vitiligo induction. But injection of WT fibroblasts alone into the tail skin dermis of IFNGR1 KO mice results in local CD8 T cell aggregation after vitiligo induction. Therefore, IFN-γ responsive fibroblasts from dermis may be used to result in local CD8 T cell aggregation and activation for vitiligo patients. IFN-γ response up-regulated fibroblasts from dermis may be used to result in local CD8 T cell aggregation for vitiligo patients.

In the sixth place, the present invention provides a method for screening candidate drugs for treating vitiligo using the said animal model.

In the seventh place, the present invention provides a method for evaluating the therapeutic effects of vitiligo using the said animal model.

In the eighth place, the present invention provides a method for prognosis evaluation of vitiligo using the said animal model.

In the ninth place, the present invention provides a use of the said animal model for screening candidate drugs for treating vitiligo of animals including human beings.

In the tenth place, the present invention provides a method for distinguishing disease states of vitiligo using single cell RNA-seq analysis.

Progressive state vitiligo skin contains more CD8+ cytotoxic T cells that express significant amount of IFNG compare to quiescent state patients and healthy donors. Correspondingly, melanocytes in progressive state vitiligo skin up regulate genes involved in immune response, especially response to IFN-γ.

Classic IFN-γ signaling activation involves Janus Kinases JAK1 and JAK2, which phosphorylate STAT1 and enable its transcription factor activity (ref). So pSTAT1 staining is used as the readout for IFN-γ responsive cells.

The vitiligo skin is divided into three regions based on skin pigmentation and T cell infiltration: depigmented lesion region, T cell infiltrated region (TIR) and adjacent pigmented perilesion region. The pSTAT1+IFN-γ responsive cell density is significantly higher in TIR compared to lesion region and perilesion region. Importantly, the density of CD8+ T cells positively correlates with the density of pSTAT1+ cells. The spatial co-distribution pattern of CD8+ T cells and IFN-γ responsive cells in patient skin is consistent with the single cell RNA-seq analysis. Therefore, the regional response to T cell secreted IFN-γ correlates with progressive disease state.

The recent development of single-cell RNA sequencing has deepened our understanding of the cell as a functional unit, providing new insights based on gene expression profiles of hundreds to hundreds of thousands of individual cells, and revealing new populations of cells with distinct gene expression profiles previously hidden within analyses of gene expression performed on bulk cell populations. However, appropriate analysis and utilization of the massive amounts of data generated from single-cell RNA sequencing experiments are challenging and require an understanding of the experimental and computational pathways taken between preparation of input cells and output of interpretable data. In this review, we will discuss the basic principles of these new technologies, focusing on concepts important in the analysis of single-cell RNA-sequencing data. Specifically, we summarize approaches to quality-control measures for determination of which single cells to include for further examination, methods of data normalization and scaling to overcome the relatively inefficient capture rate of mRNA from each cell, and clustering and visualization algorithms used for dimensional reduction of the data to a two-dimensional plot.

The invention encompasses all combination of the particular embodiments recited herein.

### BRIEF DESCRIPTION OF THE DRAWINGS

FIG. 1 shows that single cell RNA-seq analysis of vitiligo patient skin reveals distinct disease states and associated signaling signatures. Scale bar, 50 ™. Data reflect mean±SD from 5 vitiligo patients in each stage and 5 healthy donors. **p<0.01, n.s. not significant by t-test.

FIG. 2 shows that regional response to IFN-γ correlates with CD8+ T cells infiltration in vitiligo patient skin. Data reflect mean±SD from 6 human vitiligo biopsies. ***p<0.001, ****p<0.0001 by t-test.

FIG. 3 shows vitiligo mouse model revealing that response to IFN-γ signaling is required for local CD8+ T cell aggregation and cytotoxic activity in skin. Scale bar, 500 μm. Data reflect mean±SD from at least 6 mice. ***p<0.001, ****p<0.0001; n.s. not significant by t-test.

FIG. 4 shows that IFN-γ responsive skin dermal cells locally recruit and activate CD8+ T cell cytotoxic activity. Data reflect mean±SD from at least 6 independent host mice per group. ***p<0.001; n.s. not significant by paired t-test.

FIG. 5 shows that IFNGR1-Jak1-Stat1 signaling axis in dermal fibroblasts is necessary for local CD8+ T cell aggregation and activation in autoimmune skin. Data reflect mean±SD from 3 independent mice. ***p<0.001; n.s. not significant by t-test. For shRNA knockdown experiments, data were collected from at least 3 independent mice for each shRNA, and 2 shRNA were used for each gene.

FIG. 6 shows that IFN-γ responsive fibroblasts are sufficient to recruit CD8+ T cells through secreted cytokines. Data reflect mean±SD from 3 independent experiments with technical triplicates for each experiment.

FIG. 7 shows that intrinsic IFN-γ response differences of anatomically distinct human fibroblasts correlate with regional disease variations.

FIG. 8 shows FACS profile and skin sample information of vitiligo patients and healthy donors.

FIG. 9 shows single cell RNA-seq analysis of different cell populations from vitiligo patients and healthy donors.

FIG. 10 shows analysis of vitiligo mouse model.

FIG. 11 shows analysis of graft and vitiligo induced mice.

FIG. 12 shows analysis of in vivo dermal fibroblast specific knockout and knockdown experiments.

FIG. 13 shows analysis of mouse fibroblasts after in vivo intradermal fibroblasts injection, in vitro fibroblasts IFN-γ treatment and in vivo vitiligo induced expression changes in fibroblasts.

FIG. 14 shows that human skin dermal fibroblasts demonstrate intrinsic regional differences in response to IFN-γ.

### DESCRIPTION OF PARTICULAR EMBODIMENTS OF THE INVENTION

The descriptions of particular embodiments and examples are provided by way of illustration and not by way of limitation. Those skilled in the art will readily recognize a variety of noncritical parameters that could be changed or modified to yield essentially similar results.

### Experimental Model and Subject Details

Human Specimens

Seven male and three female patients who were pathologically diagnosed with vitiligo were enrolled in this study. Their ages ranged from 6 to 55, with a median age of 24. Among these patients, four were diagnosed at active stage, while the else six were not sure the stage. All the patients were newly diagnosed with vitiligo and none of the patients had received chemotherapy or UVB treatment. A 10 mm² spindle-like biopsy was taken from each patient in the junction region between depigmented lesion region and pigmented perilesional region. Biopsy with the same size was also taken from 5 healthy donors with other surgical operation as control. The clinical characteristics of these patients and healthy donors were summarized in Table 1. This study was approved by the Ethics Committee of Beijing Hospital and Ethics Committee of National Institute of Biological Science, Beijing. All patients in this study provided written informed consent for sample collection and data analyses.

TABLE 1

| | Clinical Information | | | | | |
|---|---|---|---|---|---|---|
| | | | | | Clinical | Cell |
| | | Gender | Age | Position | Diagnosis | Number |
| Healthy | 01 | Male | 78 | Knee | NA | 1941 |
| Donor | 02 | Female | 41 | Hip | NA | 3358 |
| | 03 | Female | 18 | Face | NA | 3403 |
| | 04 | Male | 33 | Chest | NA | 7768 |
| | 05 | Female | 41 | Shoulder | NA | 6223 |
| Patient | 01 | Male | 24 | Left arm | P | 1421 |
| | 02 | Male | 24 | Face | P | 1224 |
| | 03 | Male | 16 | Hip | P/Q | 4556 |
| | 04 | Male | 27 | Back | P/Q | 4667 |
| | 05 | Male | 9 | Head | P/Q | 1238 |
| | 06 | Female | 6 | Neck | P | 2629 |
| | 07 | Male | 28 | Shoulder | P/Q | 4319 |
| | 08 | Female | 55 | Shoulder | P/Q | 4123 |
| | 09 | Male | 36 | Waist | P/Q | 2954 |
| | 10 | Female | 39 | Vulva | P | 3011 |

Mice

Mice were bred and maintained in NIBS specific pathogen-free facility in accordance with the Guide for the Care and Use of Laboratory Animals of the National Institutes of Biological Sciences (NIBS). All mice used in experiments were socially houses under a 12 hrs light/dark cycle with free access to food and water.

C57BL/6 mice were purchased from Charles River Laboratories. IFNGR1 KO mice (Stock NO: 003288) were kindly provided by Dr. Feng Shao. OT-1 mice (Stock NO: 003831) were kindly provided by Dr. Liang Chen. Pdgfra-CreER (Stock NO: 018280) mice, IFNGR1 flox (Stock NO: 025394) mice, and Rosa-stop-mTmG (Stock NO: 007676) mice were from Jackson laboratories.

Cell Culture

All the cell lines were cultured at 37° C. in a cell incubator with 5% CO₂. B16F10 cells (ATCC, CRL-6475) were maintained in DMEM (GIBCO) medium supplemented with 10% (v/v) FBS (GIBCO) and 1% (v/v) Pen-strep (Invitrigen). Primary mouse fibroblast were maintained in DMEM supplemented with 10% (v/v) FBS and 1% (v/v) Pen-strep. 293FT cells (Thermo Fisher Scientific, Cat #R70007) used for virus package were maintained in DMEM medium supplemented with 10% (v/v) FBS, 1% (v/v) Pen-strep, 1% (v/v) L-glut (Lonza), 1% (v/v) 100 mM Sodium Pyruvate (Lonza), 1% (v/v) 7.5% sodium bicarbonate (Lonza), and 500 mg/mL G418 (Lonza).

Method Details

Single Cell Collection

Single cell collection started within 3 hrs after skin biopsy collection. Subcutaneous fat was carefully removed. Skin tissue was placed dermis side down in the 6 mL 2.4 U/mL disease solution at 37° C. at 80 rpm for 70 min. Epidermis with hair follicle was carefully separated from the dermis. The separated epidermis was then placed inner side down in 6 mL 0.25% Trypsin at 37° C. for 10 min. An additional 6 mL of 5% FBS media was then added to neutralize trypsin. The epidermal single cell suspension was obtained by repeatedly aspiration and dispensing with 1 mL pipette 10 times, and then filtered with strainers (70 mm followed by 40 mm). Cells were then stained with APC-CD45 antibody (1:300) and PE-CD117 antibody (1:300) for 15 min to detect immune cell and melanocyte, and then washed. The dermis part was placed in 10 mL 2 mg/mL collagenase (Sigma-Aldrich, C2674) at 37° C. and 80 rpm for 1 h. An additional 10 ml of 5% FBS media was then added. Single cell suspensions were obtained by repeatedly aspiration and dispensing with pipette 10 times. The cells were then filtered with strainers (70 mm followed by 40 mm). Cells were then stained for 15 min with APC-CD45 (1:300) for immune cell detection and then washed.

Based on FACS analysis, single cells of different subtypes, including immune cells (CD45+) from both epidermis and dermis, melanocytes (CD117+) from dermis, other dermal niches from dermis (CD45−), keratinocyte (CD45−, CD117−) from epidermis were sorted into collection buffer, which contains 0.04% BSA in PBS to minimize cell losses and aggregation. All the immune cells, melanocytes, dermal cells and the same number of keratinocytes as all three were pulled together for next step single cell analysis.

Vitiligo Mouse Model Induction

9 weeks female C57BL/6 mice were intradermally inoculated 2×10⁵ B16F10 melanoma cells in the right flank of dorsal skin. The mice were then treated with anti-CD4 antibody purchased from BioXcell (West Lebanon, NH, USA) intraperitoneally on Days 4 and 10, as previously described. Only mice that developed primary tumors were used for further analysis. Primary tumors were surgically removed on Day 12. Spontaneous tumor metastases were not observed with this B16F10 cell line, and mice with recurrent primary tumors after surgery were not used for further study. Back skin white hair appeared 2 weeks after surgery, initiating at the right flank where the primary tumor had been removed and progressed to the whole back in 10 months. About 60% of mice develop vitiligo within 30 days after induction and the remaining 40% maintain unaffected appearance.

CD4 and CD8 Depletion Antibody Administration

CD4 antibody for cell depletion was purchased from BioXcell (West Lebanon, NH, USA). CD8 antibody for cell depletion was a gift from Jianhua Sui Lab at National institute of Biological Sciences, Beijing.

Anti-CD4 (GK1.5) and Anti-CD8 (2.43) were administered i.p. in doses of 10% g/g mouse body weight. More than 99% depletion of target populations was confirmed by flow cytometry. CD4 antibody was administered on day 4 and day 10 after B16F10 inoculation. CD8 antibody was administered every fourth day after the tumor was removed.

Tail to Back Graft

For tail to back skin graft, 4-6 weeks full thickness female tail skins were removed, flattened and cut into square with 1 cm side. Only the one third part adjacent to the base of the tail was used as donor skin. Donor skin pieces were placed onto the back of anesthetized 8 weeks female recipient mice with indicated genotype, with each recipient receiving a WT and KO graft. Grafts were secured by sterile gauze and elastic bandages, which were removed after healing (8-10 days). Sex-unmatched donor skin would result in skin necrosis in 20 days. The skin adjacent to tail tips was so narrow and usually lost during wounding healing.

Genetic Labeling and Conditional Knockout

For CreER activation, Tamoxifen was dissolved in sunflower oil with 10% ethanol. For labeling, pdgfra-CreER; mTmG mice receive a single intraperitoneal (i.p.) injection of 100 μL 10 mg/mL Tamoxifen solution. Sample was taken 2 days after injection for analysis. For conditional knockout, pdgfra-CreER; ifngr1 fl/fl mice receive intraperitoneal (i.p.) injection of 200 μL 10 mg/mL Tamoxifen solution in 7 consecutive days at 6 weeks. Knock out efficiency was validated by cell type specific qPCR.

Immunofluorescence Staining

For section staining, tissues were embedded in OCT compound, frozen, cryosection (20-30 μm) and fixed for 10 min in 4% paraformaldehyde in PBS. Sections were washed in PBS overnight and then permeabilized for 20 min in 0.3% H₂O₂ in Methanol at −20° C. and blocked for 1 hr in a solution of 2% normal donkey serum, 1% BSA, and 0.3% Triton in PBS at Room temperature. The following antibody were used: anti-hCD45, anti-human Tyr, anti-human KRT14, anti-human DCT, anti-human CD8, anti-human pSTAT1, anti-human pdgfra, anti-human CD31, anti-human aSMA, anti-human CD11c, anti-human Langerin, anti-human CD3e, anti-human CD8a, anti-mouse DCT, anti-mouse pdgfra, anti-mouse CD45, anti-mouse CD31, anti-mouse aSMA, anti-mouse pSTAT1, anti-mouse KRT14. The signal of human pSTAT1, human CD8a, human pdgfra, human CD11c, human Langerin, human CD3e, mouse pdgfra, mouse pSTAT1 was amplified by ABC Kit and TSA Kit.

For whole-mount staining, the entire tail skin was harvested and flattened. Tail skin was cut into 7-8 square pieces and the central 3-4 pieces were placed in 20 mM EDTA solution at 37° C. at 80 rpm for 1.5 hrs. Epidermis was quickly removed from dermis in posterior-anterior direction with fine-tipped tweezer. This would keep most of the hair follicle in the dermis part. A few of hair follicles in the epidermis were removed by tweezer. Epidermis was flattened and fixed for 10 min in 4% paraformaldehyde in PBS. Whole-mount skin was washed in PBS overnight and then permeabilized for 20 min in 0.3% H₂O₂ in Methanol at −20° C. and then blocked for 1 hr in a solution of 2% normal donkey serum, 1% BSA, and 0.3% Triton in PBS at Room temperature. The following antibody were used: anti-mouse CD8a, anti-mouse DCT.

Imaging and Images Processing

Tissue and section samples were imaged on a Nikon A1-R confocal microscope.

Images were acquired using a 20×0.75 objective lens for representative pictures and 10×0.5 objective lens for quantitative pictures. Z-stacks were acquired at a resolution of 1024×1024, or 512×512. Microscopy data was analyzed using Imaris software with the 3D visualization module (Bitplane). RBG images were assembled in Adobe Photoshop CS3 and panels were labeled with Adobe Illustrator CS6.

To quantify the CD8+ T cell infiltration region and melanocyte remaining region, those regions containing fluorescent signals of T cells and melanocytes were converted to digital information and then quantified by the “Surface” function in Imaris software.

To describe the distribution and density of CD8+ T cells and melanocytes, fluorescent signals of T cells and melanocytes were converted to digital information by the “spot” function in Imaris software. Position coordinates of each spots was analyzed with _smooth scatter_package in R software to present the cell distribution and density. In addition to this, position information was analyzed with DBSCN package in R software to present the T cell clone size the T cell number in each clone.

To quantify the number of RFP+ fibroblast, melanocyte and CD8+ T cells in each scale, fluorescent signals of these cells were converted to digital information by the “spot” function in Imaris software. Cell number in each scale was quantified in Python.

Fibroblast Transplantation Assay

For fibroblast transplantation to the adult tail skin, the entire back skin of new born female mice was harvested, flattened and placed dermis side down on the 6 mL 2.4 U/ml Dispase solution at 37° C. at 80 rpm for 60 min. Epidermis was tightly removed from dermis. This would keep most of the hair follicle in the epidermis part. The dermis part was placed in 10 mL 2 mg/mL collagenase. (Sigma-Aldrich, C2674) at 37° C. at 80 rpm for 1 h. An additional 10 ml of 5% FBS media was then added. Single cell suspensions were obtained by repeatedly aspiration and dispensing with Finnpipette 10 times. The cells were then filtered with strainers (70 mm followed by 40 mm).

The fibroblasts were then expanded and cultured in DMEM (GIBCO) medium supplemented with 10% (v/v) FBS (GIBCO), 1% (v/v) Pen-strep/L-glut (Lonza), and 1% anti-biotic anti-myotic.

These primary new-born mouse fibroblasts were then infected with lentivirus containing LV-H2BRFP. RFP-labeled fibroblasts were then injected into the tail skin dermis of 8 weeks adult female WT or IFNGR1 KO mice. A total of 3×10⁶ fibroblasts at concentration of 10⁵/μL were intradermally injected to 3 sites per tail (10 μL per site, the interval of sites is 1 cm). Mice were left for 3 days before vitiligo induction.

Fluorescence-Activated Cell Sorting (FACS)

For isolation of immune cell and melanocyte from tail skin epidermis, the entire tail skin was harvested and flattened. Tail skin was cut into 7-8 square pieces and placed dermis side down on the 6 mL 2.4 U/ml Dispase solution at 37° C. at 80 rpm for 45 min. Epidermis was quickly removed from dermis in posterior-anterior direction with fine-tipped tweezer. This would keep most of the hair follicle in the dermis part. A few of hair follicles in the epidermis were removed by tweezer. The removed epidermis was placed inner side down to float on 6 mL TrypLE solution at 37° C. for 10 min. An additional 6 ml of 5% FBS media was then added.

Single cell suspensions were obtained by repeatedly aspiration and dispensing with Finnpipette 10 times. The cells were then filtered with strainers (70 mm followed by 40 mm). Cells were then stained for 15 min with Alex647-CD8 antibody (1:300) and FITC-CD45 (1:300) for CD8+ T cell detection, or Alex647-CD117 antibody (1:300) for melanocyte detection and then washed.

For the isolation of fibroblast and endothelial cell from tail skin, the entire tail skin was harvest and flattened. Tail skin was cut into 7-8 square pieces and placed dermis side down on the 6 mL 2.4 U/ml Dispase solution at 37° C. at 80 rpm for 60 min. Epidermis was tightly removed from dermis in anterior-posterior direction with fine-tipped tweezer. This would keep most of the hair follicle in the epidermis part. A few of hair follicles in the dermis were pulled out by tweezer. The dermis part was placed in 10 mL 2 mg/mL collagenase. (Sigma-Aldrich, C2674) at 37° C. at 80 rpm for 1 h. An additional 10 ml of 5% FBS media was then added. Single cell suspensions were obtained by repeatedly aspiration and dispensing with Finnpipette 10 times. The cells were then filtered with strainers (70 mm followed by 40 mm). Cells were then stained for 15 min with Alex647-CD31 antibody (1:300) FITC-CD45 (1:300) and then washed.

DAPI was used to exclude dead cells. Cell analysis and isolations were performed on BD AriaII sorters equipped with FACSDiva software (BD bioscience). FACS analyses were performed using LSII FACS Analyzer (BD bioscience) and then analyzed with FlowJo software (FlowJo LLC).

Letivirus Vector Construction, Production and Injection

Lentivirus expressing short hairpin RNAs were injected with an insulin syringe into P1 tail skin dermis. Vitiligo induction was starts at 9 weeks and whole mount staining was performed 33 days later.

For knock down of IFNGR1, JAK1, and STAT1, shRNA lentivirus constructs were obtained from the RNAi consortium (TRC) mouse lentivirus library. shRNA was then subcloned into LV-RFP. Sequences of individual shRNA used in experiments are listed above. For the knock down of relative genes in dermis, high titer lentivirus was produced as previously described. 10 μL high titer lentivirus (>5×10⁸ cfg/mL) was intradermally injected using insulin syringes into base of the tail. Vitiligo induction starts at 9 weeks after birth. Samples were collected to perform whole-mount staining at day26 after vitiligo induction.

In Vitro Transwell T Cell Migration Assay

Transwell migration of lymphocytes was performed with mature CTLs and concentrated fibroblast conditioned medium. In brief, splenocytes isolated from OT-1 mice were stimulated with OVA257-264 for 3 days in the presence of 10 ng/mL IL2. Cells were centrifuged and cultured in fresh medium containing 10 ng/mL IL2 for 1 more day, after which most of the cells in the culture were CTLs. To measure CD8+ T cell cytotoxicity, B16F10 expressing OVA peptide was mixed in the killing medium at the ratios of 1:1. After 6 hs, the cytotoxic efficiency was confirmed by B16F10 cell survival.

To acquire fibroblast conditioned medium, primary fibroblasts from newborn mice back skin was treated with 1000 U/mL IFN-γ containing DMEM for 6 hrs at 37° C. The concentration and duration of IFN-γ was previously validated to acquire the condition with remarkable IFN-γ response. The medium was then concentrated (1×, 5×, 10×, 25×) for chemoattractants. Concentrated DMEM or IFN-γ contained DMEM were used as control.

RNA Isolation and Real-Time PCR

Total RNAs were isolated from FACS-sorted cells with Trizol followed by extraction using Direct-Zol RNA mini-prep Kit (Zymo research). For cDNA synthesis, equal amounts of RNA were reverse-transcribed by Oligo-dT (Vazyme, R222-01). Expression levels were normalized to the expression of PPIB. Real time PCR was conducted using a CFX96TM Real-Time system (Bio-RAD) with Power SYBRR Green PCR Master Mix (Life Technologies). All primer pairs were designed for the same cycling conditions: 10 min at 95° C. for initial denaturing, 40 cycles of 10 s at 95° C. for denaturing, 30 s at 62° C. for annealing, and 10 s at 65° C. for extension. The primers were designed to produce a product spanning exon-intron boundary in each of the target genes.

RNA-Seq

RNA from FACS-purified cells was submitted to the Novogene for quantification, RNA-seq library preparation, and sequencing. The library was sequenced on Illumina HiSeq platform using the Pair-End 150 bp sequencing strategy.

IFN-γ Treat Fibroblast In Vitro

Skin biopsy from different body positions were taken from 20 or 23-week old aborted female fetus. The epidermis and dermis were mechanically separated following 2.4 U/mL dispase treatment for 1 h at 37° C. at 80 rpm. Further digestion of dermis was performed with 10 mg/mL collagenase at 37° C. at 80 rpm for 1 h. The fibroblasts were then expanded and cultured in DMEM supplemented with 10% FBS, 1% P/S, 1% antibiotics and antimycotics. Passage 3 or 4 fibroblasts were treated with 1 U/mL recombinant IFN-γ followed by RNA-sequencing. For each region, the average FPKM value of 2 individual samples was used to estimate the gene expression level. Genes in heatmap were selected on the basis that they are at least differentially expressed in 1 of 8 positions after IFN-γ treatment (Log 2 fold change >1 and p<0.01).

Quantification and Statistical Analysis

Single-Cell RNA Library Construction and Sequencing

Single cell cDNA libraries have been prepared using the Chromium Single Cell 3′ Library and Gel Bead kit v2 according to the manufacturer's instructions. In brief, cell suspensions in a chip were loaded on a Chromium Controller (10×Genomics, Pleasanton, CA) to generate single-cell GEMs (gel beads in emulsion). scRNA-seq libraries were then prepared using the Chromium Single Cell 3′Gel Bead and Library Kit (P/N #120236, 120237, 120262; 10× Genomics). Qualitative analysis of DNA library was performed by an Agilent 2100 Bioanalyzer. The concentration of DNA library was measured by Qubit (Invitrogen). Libraries were sequenced on an Illumina NextSeq 500 (2×150 paired-end reads).

Single Cell Seq Data Processing

The raw sequenced reads were aligned and quantified by Cell Ranger (V1.3.1) software which was obtained from 10×Genomics (https://support.10xgenomics.com/single-cell-gene expression/software/down-loads/latest). The human hg38 assembly reference was used for analysis. The raw count matrix data was imported into R using Seurat (V2.3.2) package for further data analysis. For each of the 15 samples, we initially set up a first filter of min.cells 3 and min.genes 200 per sample. Cells with more than 5000 genes or more than 25,000 UMI were removed. We kept cells with less than 1% mitochondrial gene expression. The raw counts were normalized by a factor of 10,000 and log-transformed to obtain log (T+1) values. Variable genes were identified by fitting the mean-variance relationship and met the following criteria: 0.0125<mean of non-zero values <3 AND standard deviation >0.4.

Cell Type Classification Using t-SNE

Unsupervised clustering of cells was performed with Seurat. Dimensionality reduction was performed using principal-component analysis. The first 20 PCs were selected according to the PCA elbow plot and used for clustering with resolution parameter 0.1. Cell clusters were visualized using t-SNE plots, with all significant principal components as input. We integrated all samples data using Canonical Correlation Analysis (CCA). The shared-nearest neighbor graph was constructed on a cell-to-cell distance matrix from top 30 aligned canonical correlation vectors. The shared-nearest neighbor graph with different resolution was used as an input for the smart local moving algorithm to obtain cell clusters, and visualized with t-SNE. On the basis of differentially expressed genes, identified by Wilcoxon rank sum test, with parameters min. pct=0.25, thresh.use=1, test.use=“wilcox” On the basis of previous knowledge and consistency within the different resolutions, we selected the final number of clusters between resolutions, which included all the major cell types in the skin, resulting in 8 different clusters.

Profiling Differentially Expressed Genes within Each Cell Cluster

To identify differentially expressed genes in 2 melanocyte subtypes, we applied the FindMarkers function from Seurat to the normalized gene expression data, with following parameter: min.pct>0.25, thresh.use=0.25, The highly expressed genes in C1 melanocyte cluster were identified as C1 signature genes (fold change>2, p-value<0.01). GO analysis of C1 signature genes were performed using GO web service (http://geneontology.org). For comparing gene expression between four T cell clusters, the differentially expressed genes were selected using the threshold fold change>0.25, p-value<0.01. The gene expression levels shown in the various charts in the manuscript were plotted by packages in R.RNA-seq Alignment, Analysis and Visualization

For mouse tail fibroblast RNA-seq analysis, raw transcriptome sequence data were mapped to the mouse genome (GRCm38/mm10) using TopHat (v2.0.13) with default settings to produce a reference-guided transcript assembly. Cufflinks (v2.2.1) was used to normalize expression levels for each sample to fragments per kilobase of transcript per million mapped reads (FPKM).

Cuffdiff was used to quantify changes in gene expression between the Control WT fibroblast, Vitiligo WT fibroblast and Vitiligo IFNGR1 KO fibroblast. Genes with significantly upregulated expression level (p-value<0.01, fold change of Vitiligo WT/Naive WT>1.5, Vitiligo WT/Vitiligo KO>1.5) were chosen for further analysis. Gene ontology (GO) analysis of upregulated genes performed using GO web-service (http://geneontology.org). Differentially expressed genes were presented by “ggplot2” package in R software.

For In vitro IFN-gamma treated human fibroblast RNA-seq analysis, raw transcriptome sequence data were mapped to the human genome (hg38) using STAR (v 2.6.1a) with default settings to produce a reference-guided transcript assembly. FeatureCount (v2.2.1) was used to count reads of genes and normalize expression levels for each sample to fragments per kilobase of transcript per million mapped reads (FPKM). Differential expression analysis using the DESeq2 package. Genes with significantly upregulated expression level (p-value<0.01, fold change >2) were chosen for further analysis. Heatmap showing differentially expressed genes were presented by “pheatmap” and “ggrepel” package in R software.

### EXAMPLES

### Example 1 Single Cell RNA-Seq Analysis of Vitiligo Patient Skin Reveals Distinct Disease State and Associated Signaling Pathways

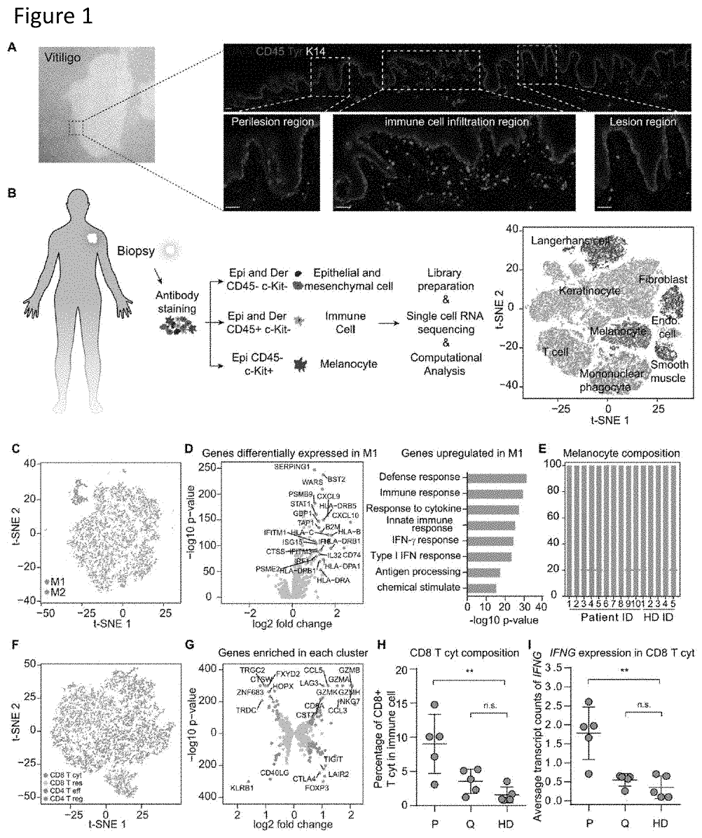

We first used immunofluorescent staining to analyze patient skin so as to investigate the cellular and molecular mechanisms leading to regional autoimmune vitiligo progression in patients. Biopsies at the border region of depigmented lesion skin and still pigmented perilesion skin were obtained from treatment-naïve vitiligo patients (FIG. 1A). FIG. 1A shows representative photo and immunofluorescent staining images of vitiligo patient skin. Photo of the lesion skin from a vitiligo patient shows depigmented skin region. In skin section immunofluorescent staining images, Tyrosinase (Tyr) staining marks melanocytes located in the basal epidermis, CD45 staining marks infiltrated immune cell and Keratin 14 (K14) staining marks keratinocytes. Enlarged images show lesion region, perilesion region and immune cells infiltrated region. Note that melanocytes are present in the perilesion region but lost in the lesion region. Infiltrated immune cells are enriched at the junction area in between. Tyr+ melanocytes are evenly distributed in the basal epidermis of perilesion region, but lost in the lesion region. Consistent with vitiligo being an autoimmune disease, we detected large amount of CD45+ immune cells infiltrating the patient skin. The depigmented lesion region contains slightly more CD45+ immune cells than the perilesion region, but majority of the infiltrated immune cells are concentrated at the junction area between the lesion region and perilesion region. This intriguing distribution pattern of CD45+ immune cells indicates certain recruitment mechanism is orchestrating the local aggregation of immune cells, which drives the expansion of depigmented region in patient skin. It was found the local aggregation pattern of CD45+ immune cells mainly at the junction area between lesion and perilesion regions.

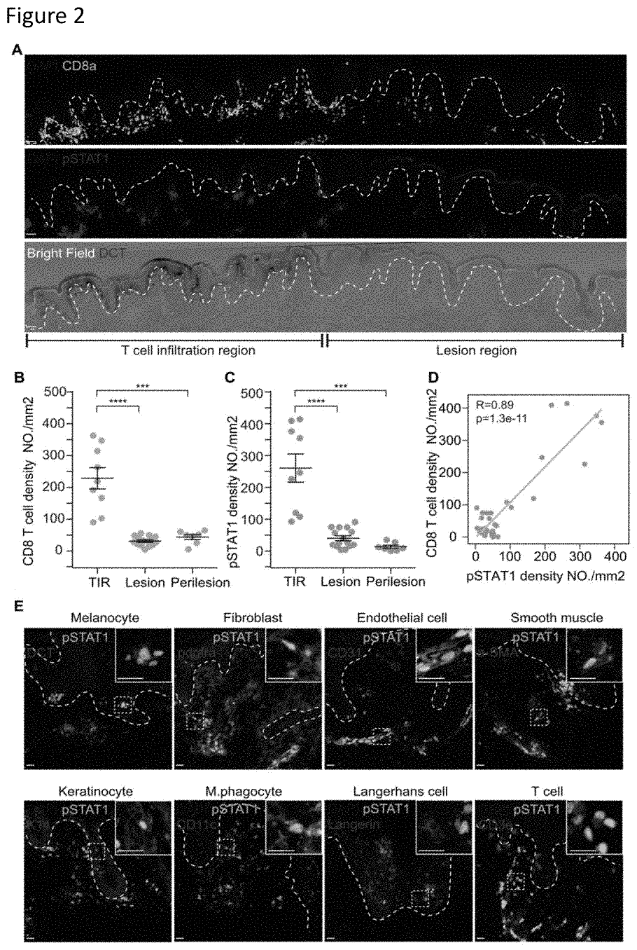

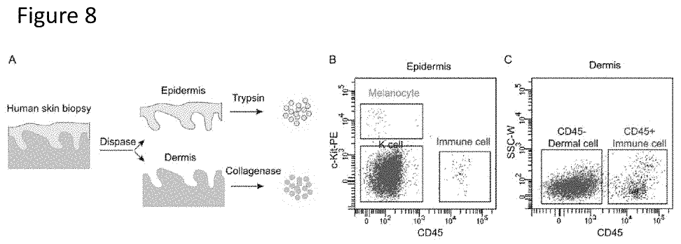

We used single cell RNA-seq to analyze all cell types present in patient skin, as controls we used cells isolated from skin biopsies of healthy donors (FIG. 2B, and FIG. 8), and tested certain skin resident cells are involved in mediating immune cells recruitment.

FIG. 2B shows quantification of CD8+ T cell density in T cell infiltration region (TIR), perilesion and lesion regions from vitiligo patients skin samples. Lesion region is defined by lack of melanocytes and skin pigmentation. Perilesional region is defined by adjacent skin area still with melanocytes and not yet infiltrated by T cells. T cell infiltration region is defined by area with enriched T cells. FIG. 8 shows FACS profile and skin sample information of vitiligo patients and healthy donors: (A) Schematic diagram of the digestion process for skin biopsies from vitiligo patients and healthy donors used in single cell-seq. Epidermis and dermis in skin biopsies were enzymatically dissociated with dispase treatment. Then skin dermis was digested with collagenase and epidermis was digested with trypsin to obtain single cell suspensions. After immunostaining, different cell types were collected using FACS; (B-C) Representative FACS profiles of different cell populations in epidermis (B) and dermis (C). In epidermis, CD45 and c-Kit were used to enrich immune cells and melanocytes; CD45−,c-Kit− cells are predominantly keratinocytes. In dermis, CD45 was used to distinguish immune cells from dermal cells; (D) Clinical information of vitiligo patients and healthy donors used in this study and number of cells sequenced in single cell analysis.

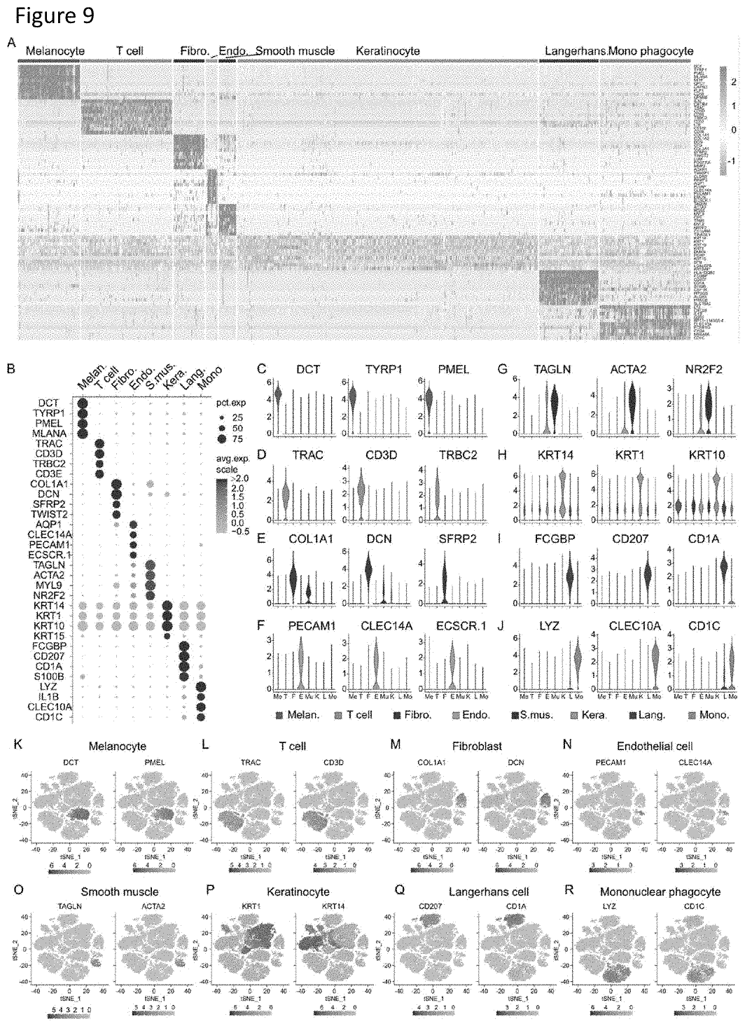

Freshly obtained skin biopsies from treatment-naïve vitiligo patients at the border region of depigmented lesion skin and pigmented perilesion skin, or from healthy donors were obtained and enzymatically dissociated to obtain single cell suspensions. After immunostaining, different cell types were isolated using FACS. All of the isolated immune cells (CD45+, c-Kit−) and melanocytes (CD45−, c-Kit+) were combined with equivalent number of niches cells (CD45−, c-Kit−) including keratinocyte and mesenchymal cells for single cell RNA-seq analysis. Unsupervised clustering of more than 50000 cells from 10 vitiligo patients and 5 healthy donors showed 8 clusters, corresponding to 8 distinct cell types defined by expression of signature genes (FIG. 1B and FIG. 9A): melanocytes (DCT, TYRP1 and PMEL); T cells (TRAC, CD3D, and TRBC2); fibroblasts (COL1A1, DCN, and SFRP2); endothelial cells (PECAM, CLEC14A, ECSCR); smooth muscle cells (TAGLN, ACTA2 and NR2F2); keratinocytes (KRT14, KRT1 and KRT10); langerhans cells (FCGBP, CD207 and CD1A); and mononuclear phagocytes including macrophage and dendritic cells (LYZ, CLEC10A, and CD1C) (FIG. 9B-9R).

FIG. 1B shows schematic diagram and overview of the single cell RNA-seq analysis of skin samples from vitiligo patients and healthy donors. Skin biopsies from vitiligo patients and healthy donors were enzymatically dissociated and then digested into single cell suspensions for FACS sorting. CD45 and c-Kit were used to enrich immune cells (CD45+, c-Kit−) and melanocytes (CD45−, c-Kit+). All the immune cells, melanocytes and equivalent number of niches cells (CD45−, c-Kit−) including keratinocyte and mesenchymal cells were pooled together to perform single cell RNA-seq. The t-SNE projection of more than 50000 cells from 10 vitiligo patients and 5 healthy donors shows 8 main cell types clusters with distinct expression profiles. Each dot represents a single cell and is colored according to annotation.

FIG. 9 shows single cell RNA-seq analysis of different cell populations from vitiligo patients and healthy donors:
(A) Heatmap analysis of signature genes in eight main cell types identified by t-SNE projection of more than 50000 cells from 10 vitiligo patients and 5 healthy donors. Unsupervised clustering performed by spectral clustering method separated the single cells into 8 sub-clusters. Differentially expressed genes of each cluster were identified using three criteria: 1. Log 2 fold change of gene expression level between certain cluster vs. others was larger than 1; 2. The p-value of differentially expressed genes in certain cluster vs. others was less than 1E10-50; 3. More than 50% of cells in the cluster express the identified differentially expressed gene unique to that cluster. Selective 10 signature genes with minimum p-value were marked alongside. Cell types were defined by the signature genes in each cluster and were marked at the topside. The underlying line color is consistent with the color used in t-SNE projection of different cell types in FIG. 1B;
(B) Dotplot analysis of the top 4 signature genes for each cell type. Genes enriched in each cell type were marked alongside. The predicted cell types were marked at the top: melanocyte, T cell, Fibroblast, endothelium cell, smooth muscle cell, keratinocyte, langerhans cell, and mononuclear phagocyte. The size of each circle depicts the percentage of cells in the subtype in which the marker was detected, and its color depicts the scaled average transcript count in expressing cells;
(C-J) Violin plots analysis showing the expression profile of 3 signature genes in each cluster: (C) Melanocyte, (D) T cell, (E) Fibroblast, (F) Endothelial cell, (G) Smooth muscle cell, (H) Keratinocyte, (I) Langerhans cell, (J) Mononuclear phagocyte. The x-axis represents different cell types as marked in the lowest panel. The y-axis represents the log-transformed, normalized gene expression level. The color of each plot depicts the predicted cell type Information.
(K-R) Feature plot analysis showing the expression patterns of 2 signature genes for each cluster in all cells: (K) Melanocyte), (L) T cell, (M) Fibroblast, (N) Endothelial Cell, (O) Smooth muscle cell, (P) Keratinocyte, (Q) Langerhans cell, (R) Mononuclear phagocyte. The color depicts log-transformed, normalized gene expression level.

Among all these identified cell types in skin, we first analyzed the transcriptional profile of melanocytes, the target cell of autoimmune attack in vitiligo skin (FIG. 1C). The t-SNE clustering revealed 2 melanocyte sub-clusters (termed M1 and M2) when all melanocytes from vitiligo patients and healthy donors were analyzed together. Interestingly, cells in the M1 cluster were almost all derived from vitiligo patient skin. We then analyzed genes enriched (>2 fold, p<0.01) in melanocytes in M1 cluster compared to all the other melanocytes from vitiligo and healthy skin in M2 cluster (FIG. 1D). Functional annotation analysis revealed majority of these genes are involved in immune response, especially response to interferon gamma (IFN-γ). Since a lot of melanocytes derived from vitiligo patient skin do belong to the M1 cluster, there are clearly heterogeneities among melanocytes from different patients. We used the single cell RNA-seq data to quantify melanocyte composition in each patient and healthy donor (FIG. 1E). Melancoytes from patients 1-5 are predominantly composed of cells in M1 cluster defined by strong immune response. Patients 6-10 have similar melanocytes compositions as healthy donors. Based on the melanocyte expression profile and composition, we tentatively classified patients 1-5 to be in progressive state, and patients 6-10 to be in quiescent state. The clinical information regarding disease state is often based on feedback from patients about whether or not the depigmentation areas were progressively expanding prior to hospital visits. So definitive classification of progressive or quiescent disease states usually requires immunohistochemical analysis of skin biopsies.

FIG. 1C shows the t-SNE projection of melanocyte dataset from vitiligo patients and healthy donors. Unsupervised clustering performed by spectral clustering method separated melanocytes into 2 sub-clusters. Each cluster is colored according to annotation: M1 (red) and M2 (blue). Melanocytes in the M1 cluster with distinct expression pattern are predominantly derived from vitiligo patient skin.

FIG. 1D shows Volcano plot and GO analysis of genes enriched in melanocytes from M1 cluster compared to all the other melanocytes in vitiligo and healthy skin. Red dots in volcano plot denote genes >2 fold upregulated (p<0.01) in M1 cluster. Functional annotation analysis reveals majority of these genes are involved in immune response, especially response to interferon gamma (IFN-γ).

FIG. 1E shows melanocyte composition in each patient and healthy donor. Color annotations correspond to the 2 melanocyte clusters derived from (C). Melancoytes from patients 1-5 were predominantly composed of cells in M1 cluster defined by strong immune response. Patients 6-10 have similar melanocytes compositions as healthy donors. So patients 1-5 were classified to be in progressive state, patients 6-10 were classified to be in quiescent state.

To validate our disease state classifications based on single cell RNA-seq analysis of melanocytes, we next analyzed the transcriptional profile of T cells, the major cell type responsible for autoimmune attack of melanocytes in vitiligo skin (FIG. 1F). We identified 4 major T cell sub-clusters from single cell data: the CD8+ cytotoxic T cells specifically expressed marker genes associated with cytotoxic activity, such as GZMA, GZMB, and CCL5; the CD8+ resident T cell cluster was characterized by high expression of TRGC, TRDC and ZNF683 genes; the CD4 effector T cell cluster was characterized by specific expression of CD4 and CD40LG; and the CD4 regulatory T cell cluster showed CTLA4, Foxp3, and TIGIT expression (FIG. 1G). CD8+ cytotoxic T cells have been shown to be able to eliminate melanocytes in experimental mouse vitiligo model (ref). So next we compared the percentage of CD8+ cytotoxic T cells in total immune cells from patients and healthy donors. In patients that we tentatively classified to be in progressive state, there are statistically significantly more CD8+ cytotoxic T cells compared to patients classified to be in quiescent state and healthy donors (FIG. 1H). In addition to this, CD8+ cytotoxic T cells from progressive state patients express significantly more IFNG compare to quiescent state patients and healthy donors (FIG. 1I). These results are consistent with our transcriptional profile analysis of melanocytes and disease state classifications.

FIG. 1F shows the t-SNE projection of T cells from vitiligo patients and healthy donors. Unsupervised clustering performed by spectral clustering method separated T cells into 4 sub-clusters. Each cluster is colored and annotated based on signature gene expression pattern: CD8 cytotoxic T cell (red), CD8 resident T cell (green), CD4 effector T cell (blue), and CD4 regulatory T cell (purple).

FIG. 1G shows Volcano plot of genes differentially expressed in each T cell sub-clusters. Colored dots in volcano plot denote genes >2 fold upregulated (p<0.01) in each of the 4 clusters.

FIG. 1H shows percentage of CD8 cytotoxic T cells in total immune cells from patients and healthy donors. Patients in progressive state (P) contain statistically significantly more CD8 cytotoxic T cells compared to quiescent state (Q) patients and healthy donors (HD).

FIG. 1I shows average transcript counts of IFNG in CD8 cytotoxic T cells from patients and healthy donors. CD8 cytotoxic T cells from patients in progressive state (P) express statistically significantly more IFNG compared to quiescent state (Q) patients and healthy donors (HD).

Our single cell RNA-seq analysis of patient skin not only helped us distinguish different disease states, but also revealed major associated signaling pathways in relevant cells types. Progressive state vitiligo skin contains more CD8+ cytotoxic T cells that express significant amount of IFNG compare to quiescent state patients and healthy donors. Correspondingly melanocytes in progressive state vitiligo skin up regulate genes involved in immune response, especially response to IFN-γ.

### Example 2 Regional Response to T Cell Secreted IFN-γ Correlates with Progressive Disease State

To validate our single cell RNA-seq result in Example 2, we used immunofluorescent staining to examine the spatial distribution pattern of CD8+ T cells and IFN-γ responsive cells in patient skin. Classic IFN-γ signaling activation involves Janus Kinases JAK1 and JAK2, which phosphorylate STAT1 and enable its transcription factor activity (ref). So we used pSTAT1 staining as the readout for IFN-γ responsive cells. We tested 6 vitiligo patient skin biopsies with preliminary clinical diagnosis to be in progressive state, and found 4 of them have enrichment of CD8+ T cells at the junction area between lesion and perilesion regions. In the representative images shown in FIG. 2A, we can detect CD8+ T cells infiltrating the vitiligo skin and pSTAT1+IFN-γ responsive cells uniquely present in the same region. To quantify this spatial correlation, we divided the vitiligo skin into three regions based on skin pigmentation and T cell infiltration: depigmented lesion region, T cell infiltrated region (TIR) and adjacent pigmented perilesion region. Quantification shows the CD8+ T cell density to be ˜250/mm² in TIR, and ˜30/mm² in both lesion and perilesion regions (FIG. 2B). Then we found the pSTAT1+IFN-γ responsive cell density is significantly higher in TIR (˜250/mm²) compared to lesion region (˜50/mm²) and perilesion region (˜30/mm²) (FIG. 2C). Importantly, the density of CD8+ T cells positively correlates with the density of pSTAT1+ cells (R=0.89, p=1.3×10⁻¹¹) (FIG. 2D). The spatial co-distribution pattern of CD8+ T cells and IFN-γ responsive cells in patient skin is consistent with our single cell RNA-seq analysis. This result shows the regional response to T cell secreted IFN-γ correlates with progressive disease state.

FIG. 2A shows representative immunofluorescent staining image of CD8a, phosphorylated STAT1 and DCT in vitiligo patient skin. White dot line indicates the basement membrane of epidermis. DCT staining marks melanocytes located in the basal epidermis, CD8a staining marks infiltrated CD8+ T cell and pSTAT1 staining marks IFN-γ responsive cells. Bright field image shows depigmentated region in vitiligo skin. Scale bar, 50 μm. FIG. 2B shows quantification of CD8+ T cell density in T cell infiltration region (TIR), perilesion and lesion regions from vitiligo patients skin samples. Lesion region is defined by lack of melanocytes and skin pigmentation. Perilesion region is defined by adjacent skin area still with melanocytes and not yet infiltrated by T cells. T cell infiltration region is defined by area with enriched T cells. FIG. 2C shows quantification of pSTAT1+ cell density in T cell infiltration region (TIR), perilesion and lesion skin regions from vitiligo patients skin samples. FIG. 2D shows scatter plot and linear regression line of CD8+ T cell density vs. pSTAT1+ cell density in vitiligo patients skin samples. Infiltrated CD8+ T cell density positively correlates with the density of pSTAT1+ cells (R=0.89, p=1.3e10-11).

The distribution patterns of pSTAT1+ cells include both epidermis and dermis, indicating multiple cells types in skin respond to IFN-γ. Next we used immunofluorescent staining with antibodies against pSTAT1 and markers of 8 different cell-types to pinpoint the identities of IFN-γ responsive cells in patient skin (FIG. 2E). To summarize we detected pSTAT1 in fibroblasts (pdgfra+), melanocytes (DCT+), endothelial cells (CD31+), smooth muscle cells (a-SMA+), mononuclear phagocytes (CD11c+), and keratinocytes (K14+); but not in langerhans cells (Langerin+) or T cells (CD3e+).

FIG. 2E shows representative immunofluorescent staining image of pSTAT1+ signal in melanocytes (DCT), fibroblasts (Pdgfra), endothelial cells (CD31), smooth muscle cells (□-SMA), keratinocytes (K14), myeloid phagocytes (CD11c), Langerhans cells (Langerin) and T cells (CD3e) in vitiligo patient skin. Co-immunofluorescent staining results reveal nuclear pSTAT1+ signal present in melanocytes, fibroblasts, endothelial cells, smooth muscle cells, keratinocytes and dendritic cells; but not in the Langerhans cells and T cells. Scale bar, 10 μm.

### Example 3 Vitiligo Mouse Model Reveals that Response to IFN-γ is Required for Local CD8+ T Cell Aggregation and Activation in Skin

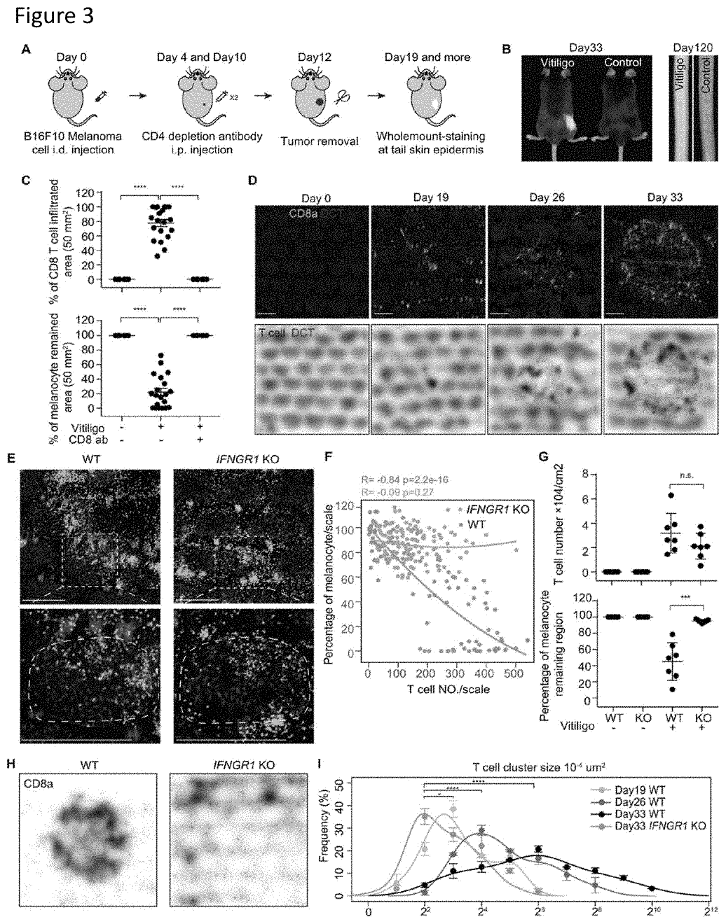

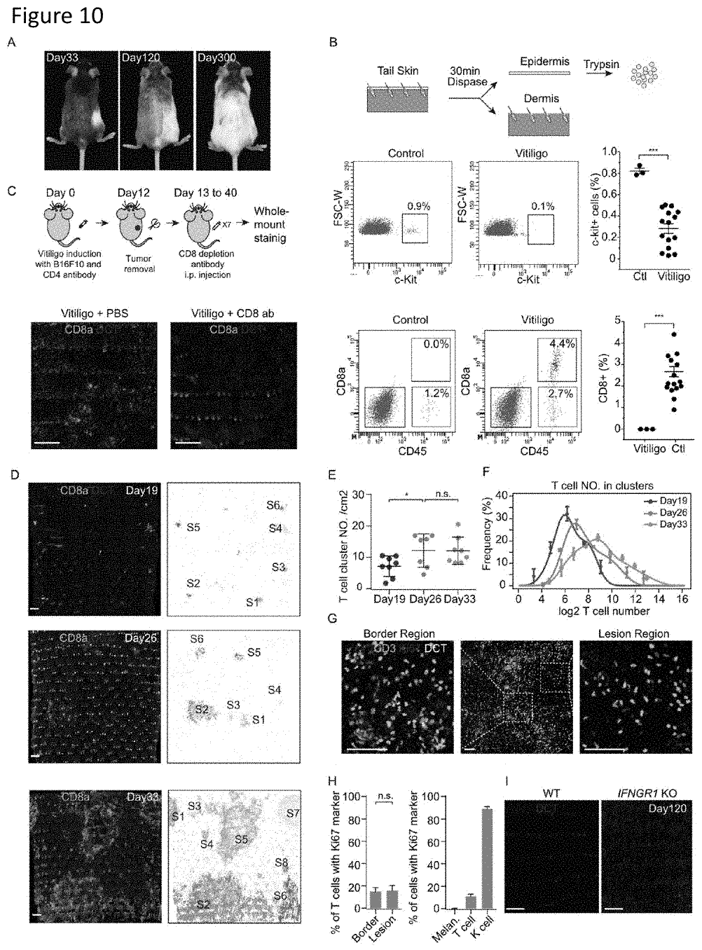

To test whether or not the cancer immunotherapy related mouse model could be used to investigate autoimmune disease mechanisms, we optimized the relevant method and developed our own protocol (FIG. 3A): First 9-week-old C57 mice are inoculated with B16F10 melanoma cells in the right flank of dorsal skin; the CD4 antibody for cell depletion purchased from BioXcell (West Lebanon, NH, USA) is injected on Days 4 and 10. The tumors are surgically removed on Day 12 to prevent tumor cells expanding and metastasizing. Because melanocytes in mouse dorsal skin are located in hair follicles but not in epidermis, so mouse tail skin is used for vitiligo analysis since it contains epidermis localized melanocytes similar to human skin. We observed at 4 weeks after induction, dorsal skin hair follicles close to the B16F10 tumor injection and surgical removal site start to show depigmentation first (FIG. 3B). Based on large number of trial and error experimental results, we find that this phenomenon is an early indicator of successful vitiligo induction. It should be noted here that only hair follicles at the tumor injection site are shaved for the procedure while the rest of the hair coat on dorsal skin is not. The first area of hair follicles entering new growth phase on the 9-week-old C57 mice is the tumor injection and removal site as a result of skin wounding. So the depigmented hair follicles in this region result from new hair growth. In order for the rest of the dorsal skin hair coat to become visually depigmented, the original pigmented hair follicles need to be completely shed while the newly formed depigmented hair follicles emerge, which takes several spontaneous hair cycles spanning almost a year (FIG. 10A). In tail skin, epidermis depigmentation becomes visually apparent at 16 weeks post induction, mainly depending on the natural turnover rate of pigmented keratinocytes on skin surface (FIG. 3B). To analyze vitiligo induction effect, we used FACS to quantify the infiltration of CD8+ T cells and loss of epidermal melanocytes in mouse tail skin (FIG. 10B). Compared to control mice tail skins that contain almost no CD8+ T cells, at Day40 after vitiligo induction vast number of CD8+ T cells infiltrated into tail skin epidermis, accompanied by significant loss of melanocytes. To functionally test whether or not the CD8+ T cells are responsible for melanocyte loss, we used CD8 depletion antibody after vitiligo induction. This results in complete block of CD8+ T cell infiltration in tail skin epidermis and rescue of melanocytes loss (FIG. 3C, FIG. 10C). So our vitiligo induction protocol efficiently triggers endogenous activated CD8+ T cells infiltrating skin that results in loss of native melanocytes located in epidermis similar to autoimmune vitiligo patients. The main experimental advantage of our vitiligo induction protocol is that it only utilizes commercially available reagents and can efficiently induce patient like vitiligo pathologies on any mice stains with genetic alterations such as knockout, conditional knockout, or transgene; hence will enable us to ask in-depth mechanistic questions.

FIG. 3A shows schematic diagram of vitiligo induction strategy in mouse model. Adult C57 background mice were intradermally inoculated with 1.5×10⁵ B16F10 cells on the right flank of dorsal skin. At days 4 and 10 later two doses of CD4 depletion antibody were injected to deplete CD4 T cells. Tumors were surgically removed on Day 12. Wholemount staining was performed at indicated time points in tail skin epidermis to analyze T cell infiltration and melanocyte loss; FIG. 3B shows representative photos of dorsal skin and tail skin depigmentation on vitiligo mouse model at indicated time points. Dorsal skin hair follicles close to the B16F10 tumor injection and surgical removal site start to show depigmentation first, at ˜4 weeks after induction. In tail skin, although T cell infiltration and melanocyte loss can be observed as early as Day19 by wholemount immunofluorescent staining, overall tail skin depigmentation only become visually apparent at 16 weeks post induction, mainly due to the natural turnover rate of pigmented keratinocytes on skin surface; FIG. 3C shows representative whole-mount immunofluorescent staining and density plot images of CD8a+ T cells and DCT+ melanocytes on the tail skin of vitiligo mouse model at Day0, Day19, Day26, and Day33 after induction. SmoothScatter density plot images indicate the distribution and density of melanocytes (red) and infiltrated CD8 T cells (blue) in whole mount images. Prior to vitiligo induction melanocytes are evenly distributed in tail skin epidermis and there are no detectible CD8+ T cells present. Starting from Day19 after vitiligo induction, the infiltration and local aggregation of CD8a+ T cell in epidermis are observed concomitant with melanocyte loss in the same region. At later time points, T cell cluster progressively expand and T cell density appears to be especially high at the border region of the clusters.

FIG. 10A shows representative photos of mice at Day33, Day120 and Day300 after vitiligo induction. Gradually the back skin hair follicle depigmentation areas expand from the B16F10 tumor injection and surgical removal site in right flank to the entire dorsal area. It should be pointed out that only hair follicles at the tumor injection site were shaved for the procedure while the rest of the hair coat at back skin was not. The original hair coat prior to vitiligo induction is pigmented, and only the new hair follicles emerged after vitiligo induction would be depigmented. Since the vitiligo induction started at 9-week old when most of the dorsal skin hair follicles are in prolonged telogen. The first area of hair follicles entering new growth phase is the tumor injection and removal site as a result of skin wounding. In order for the rest of the dorsal skin hair coat to appear completely depigmented, the original hair follicles need to be completely shed while the newly formed depigmented hair follicles emerge, which takes several hair cycles spanning almost a year; FIG. 10B shows representative whole-mount immunofluorescent staining images and density plot images of CD8+ T cells and DCT+ melanocytes in tail skin epidermis at Day19, Day26 and Day33 after vitiligo induction. CD8+ T cell immunofluorescent signals were digitally converted and analyzed by dbscan package in R. Result shows clone like T cell clusters aggregation and expansion in the epidermis; FIG. 10C shows quantification of the CD8+ T cell cluster number in skin epidermis at Day19, Day26 and Day33 after vitiligo induction. T cell cluster number was acquired and analyzed by DBSCAN package in R. Results show the number of CD8+ T cell clusters increased from Day19 to Day26 and remained unchanged from Day26 to Day33. Data reflect mean±SD from at least 6 mice.

To characterize the spatiotemporal progression pattern of vitiligo in the induced mouse model, we used wholemount immunofluorescent staining to analyze tail skin at different time points. Prior to vitiligo induction, melanocytes are evenly distributed in tail skin epidermis, and very few if any CD8+ T cells can be detected. Starting from Day19 after vitiligo induction, sparse infiltration of CD8+ T cells in epidermis and small regions of melanocyte loss can be observed. Interestingly we noticed the loss of melanocytes corresponds to the region where CD8+ T cells locally aggregate into clusters. Instead of evenly distributed in skin epidermis after infiltration, CD8+ T cells keep aggregating into small clone like clusters that expand continuously at later time points (FIG. 3D, FIG. 10D). To quantify this intriguing phenomenon we analyzed the CD8+ T cell cluster number and size in vitiligo skin at Day19, 26 and 33 after induction (FIG. 10E-10F). The clone like CD8+ T cell cluster number steadily increases from Day19 to Day26, indicating newly emerged cluster continuously form. But from Day26 to day33 the CD8+ T cell cluster number remain steady. Instead the overall cluster size increases continuously across the 3 time points; in particular large CD8+ T cell cluster not present in Day19 become frequent at Day 33. We also noticed the CD8+ T cell density are highest at the border region of T cell clusters, between the depigmented lesion skin and pigmented perilesion skin similar to the progressive state vitiligo patient skin. We did not detect local proliferation of skin infiltrated CD8+ T cells (FIG. 10G), so the local aggregation of CD8+ T cells at the border region of existing T cell clusters and the continuous expansion of cluster size result from skin infiltrated CD8+ T cells being actively recruited into regions with high CD8+ T cell density. We did not detect a higher proliferation rate of CD8+ T cells at the border than inside the T cell clusters with Ki67 staining (FIG. 10H). Since melanocyte loss is only observed within the T cell clusters, this also explains the regional expansion of depigmention in patient and mouse model vitiligo skin. Based on these observations, it was hint that there is a local positive feedback recruitment mechanism orchestrating the regional aggregation of CD8+ T cells that is essential for effective T cell cytotoxic activity. This phenomenon is analogous to quorum sensing in bacterial populations, which describes the change in gene expression in response to population density. What we observe here indicates skin infiltrated CD8+ T cells can somehow perceive the regional density of their own population and gravitate towards the high-density area.

FIG. 3D shows quantifications of CD8+ T cell density and melanocyte remaining area in control, vitiligo induced and CD8 depletion antibody treated vitiligo induced mice tail skin epidermis. Without vitiligo induction, tail skin epidermis contains no CD8+ T cells. At Day40 after vitiligo induction, vast amount of CD8+ T cells infiltrate tail skin epidermis, which can be completely blocked by treatment of CD8 depletion antibody. Correspondingly, loss of melanocyte after vitiligo induction is completely rescued by CD8 depletion antibody treatment.

FIG. 10D shows cell number distribution of CD8+ T cells in each T cell cluster as a function of vitiligo induction time points at Day19, Day26, Day33 in WT skin. Cell number in each T cell cluster is quantified by the DBSCAN and VCD package in R. Data reflect mean±SD from at least 6 mice; FIG. 10E shows schematic diagram, FACS profile and quantification of melanocyte and CD8+ T cell in tail skin epidermis of control and vitiligo-induced mice at Day33 post induction. Epidermis and dermis of tail skin were enzymatically dissociated with dispase treatment. Then skin epidermis was digested with trypsin to obtain single cell suspensions. After immunostaining of c-Kit, CD45 and CD8, percentages of melanocyte (c-Kit+) and CD8+ T cell (CD45+, CD8+) were quantified using FACS. Data reflect mean±SD from 3 mice in control group and 15 mice in experimental group. ***p<0.001 by t-test; FIG. 10F shows schematic diagram and representative whole-mount immunofluorescent staining images of CD8 depletion antibody treatment on vitiligo mouse model. Adult C57 background mice were intradermally inoculated with 1.5×10⁵ B16F10 cells on the right flank of dorsal skin. At days 4 and 10 later two doses of CD4 depletion antibody were injected to deplete CD4 T cells. Tumors were surgically removed on Day 12. From day 13-40, CD8 depletion antibodies were injected every other day for 7 dosages total. Wholemount staining was performed at Day40 to analyze T cell infiltration and melanocyte loss in tail skin epidermis. DCT staining marks melanocytes; CD8a staining marks infiltrated T cell. This result indicates CD8+ T cell depletion prevented melanocyte loss in the vitiligo tail skin epidermis; FIG. 10G shows representative whole-mount immunofluorescent staining images of DCT+ melanocytes in tail skin epidermis of vitiligo-induced WT and IFNGR1 KO mice at day120 after vitiligo induction. Even at Day120 after vitiligo induction, melanocytes in IFNGR1 KO skin still remain intact (FIG. 10I). (Scale bar, 500 μm.)

Our single cell RNA-seq and immunohistochemistry analysis of vitiligo patient skin revealed the regional response to T cell secreted IFN-γ correlates with progressive disease state. To investigate whether the IFN-γ responsive cells in skin play functional role in mediating T cell local aggregation, we used IFNGR1 KO mice to induce vitiligo. Whole mount immunofluorescent staining revealed at Day33 after vitiligo induction, both WT and IFNGR1 KO mice showed robust infiltration of CD8 T cells into tail skin epidermis (FIG. 3E). But strikingly only in WT tail skin CD8+ T cells locally aggregate into clusters leading to melanocyte loss in the same region; in IFNGR1 KO tail skin, the infiltrated CD8+ T cells uniformly distribute throughout the skin epidermis without aggregating into clusters and there is no melanocyte loss (FIG. 3E). We quantified the CD8+ T cell number and melanocytes remaining region in each scale of vitiligo induced WT and IFNGR1 KO tail skin. In WT vitiligo skin, the CD8+ T cell number negatively correlates with melanocyte cell number (R=−0.84, p=2.2×10⁻¹⁶); but in IFNGR1 KO vitiligo skin, CD8+ T cell number shows no correlation with melanocyte number (R=−0.09, p=0.27) (FIG. 3F). This quantitative result confirms the lack of cytotoxic activity of CD8+ T cells infiltrating the IFNGR1 KO tail skin. We also quantified the total number of CD8+ T cell and melanocyes remaining region in tail skin epidermis in WT and IFNGR1 KO mice with or without vitiligo induction using wholemount staining (FIG. 3G). Even though similar number of CD8+ T cells infiltrate the tail skin of WT and IFNGR1 KO mice after vitiligo induction, only in WT skin, T cell infiltration lead to melanocyte loss. This failure of infiltrated CD8+ T cells to attack melanocyte is not due to delayed activity, because even at 6 months after vitiligo induction, melanocytes in IFNGR1 KO tail skin are still intact.

FIG. 3E shows representative whole-mount immunofluorescent staining images of CD8+ T cells and DCT+ melanocytes in tail skin epidermis of vitiligo-induced WT and IFNGR1 KO mice. At Day33 after vitiligo induction in WT and IFNGR1 KO mice, tail skin epidermis show robust infiltration of CD8+ T cells. But only in WT tail skin, CD8+ T cells show local aggregation and progressive cluster expansion leading to melanocytes loss in the same region. In IFNGR1 KO tail skin, CD8+ T cells uniformly distribute without aggregating into clusters and melanocytes remain intact; FIG. 3F shows scatter plots and linear regression lines of CD8+ T cell number vs. percentage of melanocytes in each scale of vitiligo-induced WT and IFNGR1 KO tail skin. In WT vitiligo skin, CD8+ T cell number negatively correlates with melanocyte cell number (R=−0.84, p=2.2×10⁻¹⁶), but in IFNGR1 KO vitiligo skin, CD8+ T cell number show no correlation with melanocyte cell number (R=−0.09, p=0.27); FIG. 3G shows quantification of T cell number and percentage of melanocyte remaining area in WT and IFNGR1 KO skin at Day 33 with or without vitiligo induction. Even though similar number of T cells infiltrated the tail skin of in WT and IFNGR1 KO mice after vitiligo induction, only in WT skin T cell infiltration leads to melanocyte loss.

We were particularly intrigued by the different distribution pattern of CD8+ T cells in IFNGR1 KO tail skin revealed by the wholemount analysis. Smoothscatter density plot images highlight the clone like CD8+ T cell clusters in WT vitiligo skin, with T cell density particularly high at the border region. But in IFNGR1 KO vitiligo skin, CD8+ T cells show almost completely even distribution without local aggregation behavior (FIG. 3H). To quantify this drastic difference, we analyzed CD8+ T cell cluster size in tail skin of vitiligo induced WT and IFNGR1 KO mice at different time points. In WT vitiligo skin the clone like CD8+ T cell cluster sizes steadily increase from Day19 to Day33, suggesting skin infiltrated CD8+ T cells are actively recruited into existing clusters. But in IFNGR1 KO vitiligo skin the CD8+ T cell cluster size at D33 after induction is significantly smaller than those found in WT vitiligo skin from Day19 to Day33 (FIG. 3I). This result demonstrates that response to IFN-γ is essential for CD8+ T cell local aggregation and cytotoxic activity in skin.

FIG. 3H shows SmoothScatter density plot images of infiltrated T cell (blue) in tail skin epidermis of vitiligo-induced WT and IFNGR1 KO mice. Note the clone like cluster of CD8 T cells in WT skin, compared to evenly distributed CD8 T cells in IFNGR1 KO skin; FIG. 31 shows size distribution of T cell clusters as a function of vitiligo induction time points at Day19, Day26, Day33 in WT skin and at Day33 in IFNGR1 KO skin. T cell cluster size is quantified by the area of each T cell cluster.

Although we used IFNGR1 straight KO mice, the IFN-γ responsive cells mediating this effect seems to be regional in skin, because the main defect lies in the failure of T cells local aggregation, rather than the failure of T cells infiltrating skin. Since our immunofluorescent analysis of patient skin revealed T cells to be pSTAT1−, this rules out CD8+ T cells promoting self-aggregation via autocrine IFN-γ signals. There are at least 6 different cell types in patient skin that are IFN-γ responsive, ranging from keratinocytes, melanocytes, fibroblasts, endothelium cells, smooth muscle cells, to dendritic cells.

### Example 4 Skin Dermal Cells Mediate Local CD8+ T Cell Aggregation and Activation Through IFN-γ Signaling

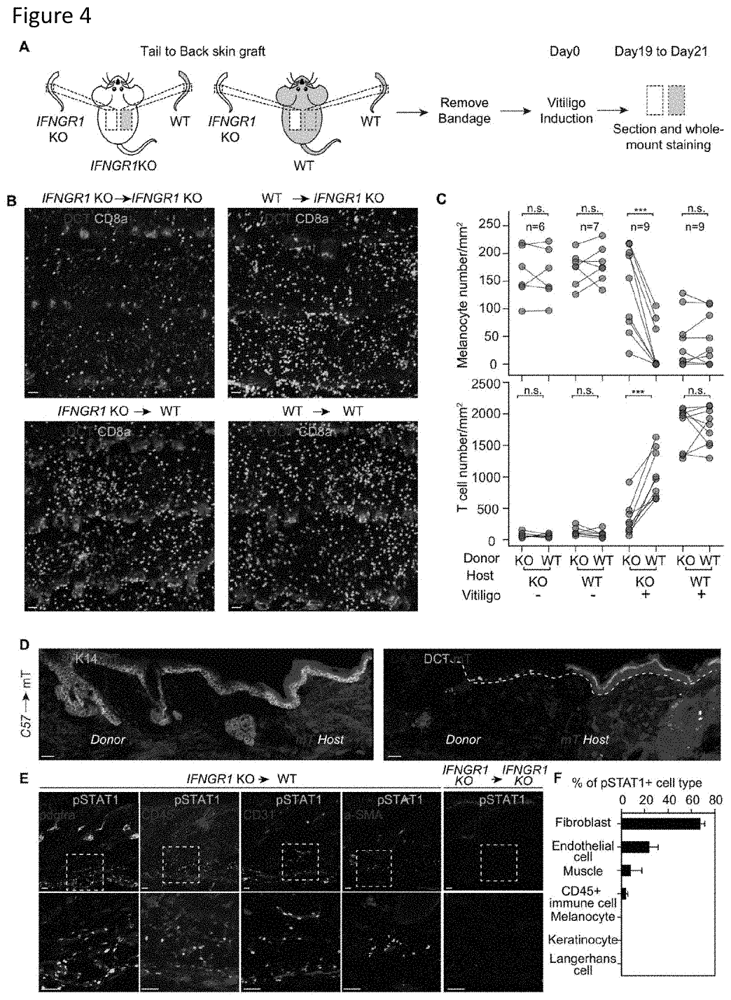

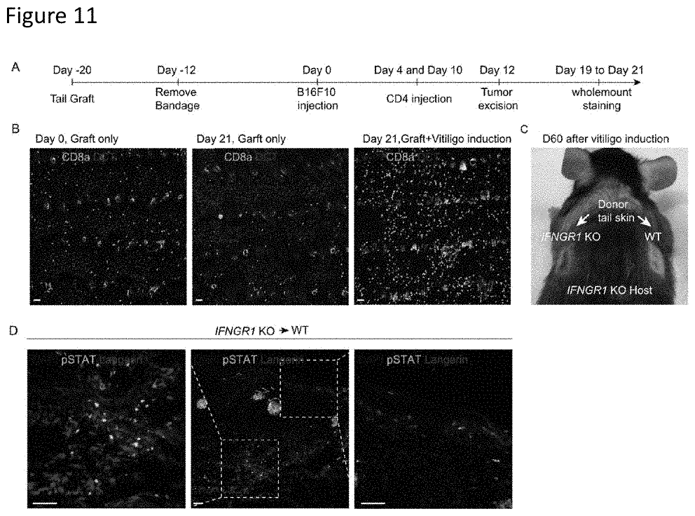

Prior to identifying which skin resident cells are responsible for orchestrating T cell local aggregation and activation upon receiving IFN-γ, first we need to rule out the possibility that the CD8+ T cells from IFNGR1 KO mice are intrinsically defective. Previous studies have shown that T cell cytotoxicity was not affected in IFNGR1 KO mice. To test whether or not in our vitiligo mouse model the same is true, we used skin graft and vitiligo induction assay to investigate if giving a WT local environment the IFNGR1 KO mice derived CD8+ T cell cytotoxic activity is normal or not (FIG. 4A, FIG. 11A). To do this we grafted full thickness tail skin from WT mice onto the back of IFNGR1 KO host mouse (WT->IFNGR1 KO graft). After vitiligo induction on the host mice, T cell infiltration and melanocyte loss in the grafted tail skin will be analyzed by wholemount immunofluorescent staining. Without vitiligo induction on the host mice, graft assay along does not result in epidermal melanocyte loss or CD8+ T cell infiltration in the grafted tail skin; only after vitiligo induction on the host mice there are large numbers of CD8+ T cell infiltrating the grafted tail skin leading to epidermal melanocyte loss (FIG. 11B).

FIG. 4A shows schematic diagram of graft and vitiligo induction assay. Full thickness tail skins from WT and IFNGR1 KO mice were grafted onto the back of host mice with indicated genotype. After vitiligo induction on host mice, grafted tail skin was analyzed for T cell infiltration and melanocyte loss using wholemount immunofluorescent staining.

FIG. 11A shows time line of the graft and vitiligo induction assay. Full thickness tail skins from 4-6 week old mice were grafted onto 8-week old host mice. B16F10 tumor injection date was denoted as Day0, so tail skin graft date was Day-20 accordingly.

FIG. 11B shows representative whole-mount immunofluorescent staining image of grafted tail skin epidermis at Day0, and Day21 indicated in (A) with or without vitiligo induction. DCT staining marks melanocytes located in the basal epithelial layer; CD8a staining marks infiltrated T cell. There are very few CD8+ T cell in the grafted skin and no obvious melanocyte loss at Day20 without vitiligo induction. But after vitiligo induction there are robust CD8+ T cell infiltration and melanocyte loss in the grafted skin.

For the WT->IFNGR1 KO graft experiment, tail skin from IFNGR1 KO mice was grafted onto the same IFNGR1 KO host on the other side of back as an internal control (IFNGR1 KO->IFNGR1 KO graft). At Day21 after vitiligo induction on the host mice, CD8+ T cells derived from IFNGR1 KO host mice robustly infiltrate the grafted WT tail skin and result in loss of WT melanocyte; but the same host derived CD8+ T cells only sparsely infiltrate the grafted IFNGR1 KO skin and do not result in loss of IFNGR1 KO melanocyte (FIG. 4B). At Day60 after vitiligo induction on the host mice, skin depigmentation on the WT->IFNGR1 KO graft can readily be observed, but the IFNGR1 KO->IFNGR1 KO graft on the same host is still pigmented (FIG. 11C). We quantified the number of infiltrated CD8+ T cell and melanocyte in the grafted skin with or without vitiligo induction on the host mice (FIG. 4C). Without vitiligo induction on host mice, there are very few CD8+ T cells in either IFNGR1 KO->IFNGR1 KO graft or WT->IFNGR1 KO graft, and the melanocyte numbers in both grafts are equivalent. After vitiligo induction on host mice, there are statistically significantly more CD8+ T cells in WT->IFNGR1 KO graft compared to IFNGR1 KO->IFNGR1 KO graft on the same host; reversely the melanocyte number is much lower in WT->IFNGR1 KO graft compared to IFNGR1 KO->IFNGR1 KO graft on the same host. This result unequivocally demonstrates that: 1. CD8+ T cell cytotoxic potential is normal in IFNGR1 KO mice; 2. Skin resident IFN-γ responsive cells are required for local CD8+ T cell recruitment and activation.

To further validate that skin resident IFN-γ responsive cells are required for local CD8+ T cell recruitment and activation, we carried out the reverse graft experiment: IFNGR1 KO->WT and WT->WT on the same host. The rationale is that if skin resident IFN-γ responsive cells are required for local CD8+ T cell recruitment and activation, then the IFNGR1 KO grafted skin won't be able to attract CD8+ T cells derived from the WT host after vitiligo induction, but the WT graft on the same host will. We were surprised to find the completely different result. CD8+ T cells derived from WT host mice robustly infiltrate both grafted WT and IFNGR1 KO tail skin and result in equivalent loss of WT and IFNGR1 KO melanocyte (FIG. 4B). Quantifications show, without vitiligo induction on host mice, there are very few CD8+ T cells in either IFNGR1 KO->WT graft or WT->WT graft, and the melanocyte numbers in both grafts are equivalent. After vitiligo induction on host mice, there are equal number of infiltrated CD8+ T cells in WT->WT graft and IFNGR1 KO->WT graft on the same host, and the melanocyte number is equally decreased in WT->WT graft compared to IFNGR1 KO->WT graft on the same host (FIG. 4C). This result seems to directly contradict our conclusion that skin resident IFN-γ responsive cells are required for local CD8+ T cell recruitment and activation. Because in the IFNGR1 KO graft where supposedly all cells are IFN-γ non-responsive, CD8+ T cell recruitment is just as efficient as in the WT graft. One explanation for this discrepancy is that WT cells from the host migrated into the grafted IFNGR1 KO tail skin, and these WT cells functionally rescued the IFNGR1 KO grafted skin in terms of recruiting CD8+ T cells.

To test this explanation, full thickness C57 tail skin was grafted to membrane-tdTomato (mT) expressing host mice (Rosa-mT), in which all cells are genetically labeled to be mT+ (FIG. 4D). Immunofluorescent staining of the junction area between grafted donor C57 tail skin and host dorsal skin of mT mice showed that host dermal cells indeed have invaded into the grafted skin dermis. But neither keratinocytes nor melanocytes show infiltration from host to grafted skin. This result supports our explanation that WT cells from the host migrated into the grafted IFNGR1 KO tail skin and functionally rescued the grafted skin; it also eliminates keratinocytes and melanocytes as the candidate cell type for this rescue effect. Since the graft experiment severed the neuron connection in intact skin, it is unlikely that neuron is the target cell we are searching for either. The robust infiltration of dermal cells from host to graft suggests that skin dermal cells are the cell type responsible for recruiting CD8+ T cells upon receiving IFN-γ. To distinguish which WT dermal cells migrate into the grafted IFNGR1 KO tail skin, we used co-immunofluorescent staining of pSTAT1 and cell type markers (FIG. 4E). In IFNGR1 KO->IFNGR1 KO graft, no pSTAT1+ signals are present in graft skin after vitiligo induction on host mice, as expected. But in IFNGR1 KO->WT graft, ectopic pSTAT1+ signals can be detected in graft skin after vitiligo induction on host mice, indicating WT cells from the host indeed migrate into the graft and respond to T cells secreted IFN-γ. Co-immunofluorescent staining results revealed nuclear pSTAT1+ signal present in fibroblasts (pdgfra+), endothelial cells (CD31+), smooth muscle cells (a-SMA+), and immune cells 9CD45+); but not in keratinocytes (K14+) or langerhans cells (Langerin+) (FIG. 4E and FIG. 11D). We quantified the distribution of pSTAT1+ in these different cell types and found ˜70% of the pSTAT1+ cells are fibroblast, ˜20% to be endothelial cells, and <10% to be smooth muscle cells or immune cells (FIG. 4F).

FIG. 4B shows representative whole-mount immunofluorescent staining of CD8+ T cells and DCT+ melanocytes on grafted tail skin after vitiligo induction on host mice. At Day21 after vitiligo induction on host mice, CD8 T cells derived from IFNGR1 KO host mice robustly infiltrated the grafted WT tail skin leading to loss of WT melanocytes, but the same host derived CD8+ T cells only sparsely infiltrated the IFNGR1 KO tail skin grafted on the same host and didn't result in loss of IFNGR1 KO melanocytes. Conversely CD8+ T cells derived from WT host mice infiltrated the grafted WT and IFNGR1 KO tail skin at similar intensity, and resulted in efficient loss of both WT and IFNGR1 KO melanocytes; FIG. 4C shows quantification of T cell number and melanocyte number on grafted tail skin with or without vitiligo induction on host mice. Donor tail skin pairs grafted onto the same host mouse were linked by lines. Genotypes of donor skin and host mice were marked; FIG. 4D shows representative immunofluorescent staining of the junction region between grafted donor C57 tail skin and host dorsal skin of membraneTomato transgenic mice. mTomato signal marks all cells from the host mice. K14 staining marks keratinocytes. DCT staining marks melanocytes that are only present in epidermis of tail skin but not in dorsal skin. Samples were taken at 20 days after graft. Note clear infiltration of host dermal cells into the grafted skin dermis. But neither keratinocytes nor melanocytes showed infiltration from host to grafted skin; FIG. 4E shows representative immunofluorescent staining image of pSTAT1+ signal in fibroblasts (Pdgfra), immune cells (CD45), endothelial cells (CD31), and smooth muscle cells (α-SMA) in grafted tail skin after vitiligo induction on host mice. In IFNGR1 KO->IFNGR1 KO graft, no pSTAT1+ signal can be detected in grafted skin after vitiligo induction on host mice. But in IFNGR1 KO->WT graft, ectopic pSTAT1+ signal can be detected in grafted skin after vitiligo induction on host mice, indicating infiltration of WT cells from the host into the grafted IFNGR1 KO skin. Co-immunofluorescent staining results reveal nuclear pSTAT1+ signal present in fibroblasts, endothelial cells, and smooth muscle cells; FIG. 4F shows quantification of the pSTAT1+ cell distribution in indicated cell types in IFNGR1 KO->WT graft after vitiligo induction on host mice. Data reflect mean±SD from 3 independent host mice. (Scale bar, 50μm)

FIG. 11C shows representative photo of the graft and vitiligo-induced mouse. Full thickness tail skins from WT and IFNGR1 KO mice were grafted onto the back of IFNGR1 KO host mouse. At Day60 after vitiligo induction on host mice, WT grafted tail skin is visually depigmented while the IFNGR1 KO grafted tail skin remains to be pigmented; (Scale bar, 50 μm)

Together our graft and vitiligo induction experiments demonstrate that since majority of the ectopic pSTAT1+ cells in IFNGR1 KO->WT graft turns out to be fibroblasts, and this leads to complete rescue of the IFNGR1 KO skin in terms of CD8+ T cell recruitment, next we used genetic experiment to determine if skin dermal fibroblast is the main cell type mediating local CD8+ T cell aggregation and activation.

### Example 5 IFNGR1-JAk1-STAT1 Signaling Axis in Dermal Fibroblasts is Required for Local CD8+ T Cell Recruitment and Activation in Autoimmune Skin

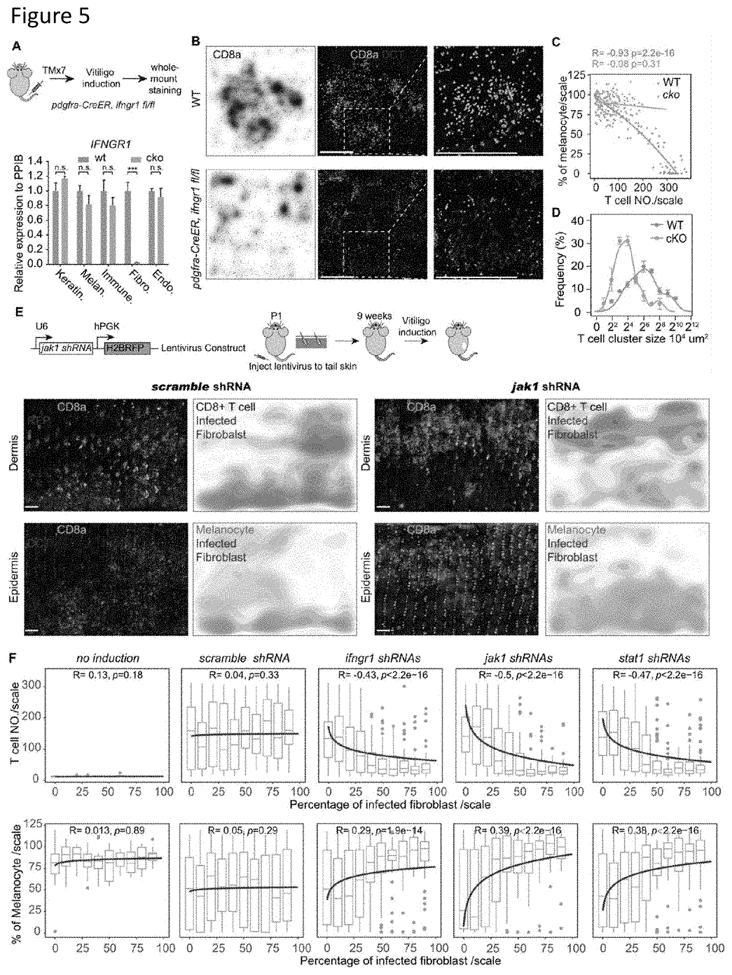

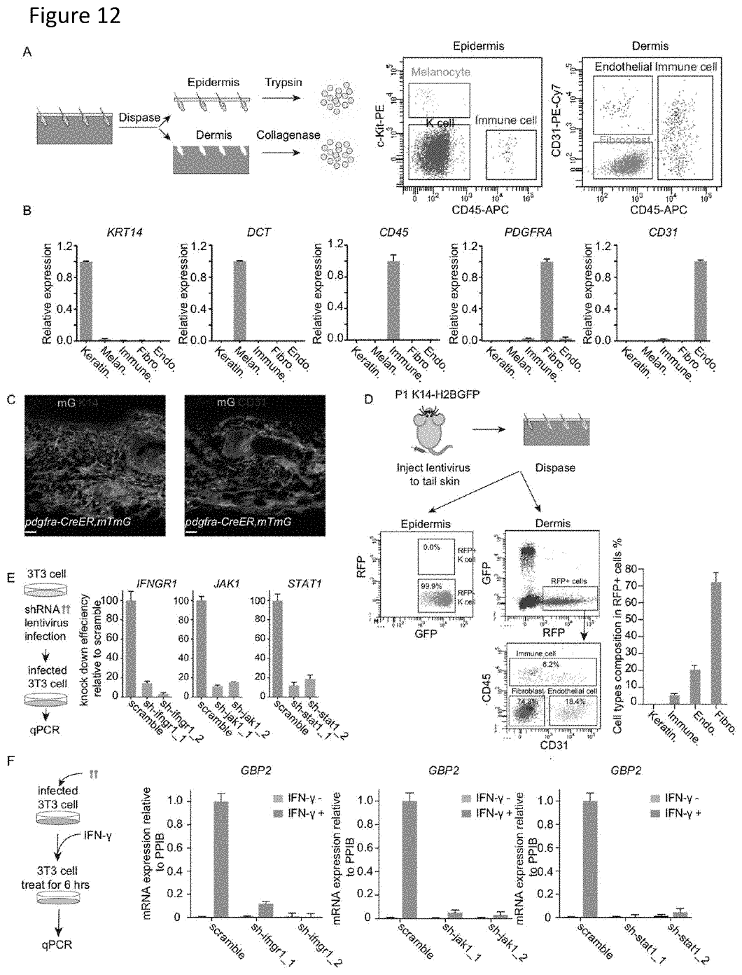

To specifically ablate IFNGR1 in fibroblast, we used the Pdgfra-CreER::IFNGR1 fl/fl mice. After tamoxifen injection from P50 to P56, FACS purified keratinocytes, melanocytes, immune cells, endothelial cells and fibroblasts from skin were used to validate the knockout specificity and efficiency (FIG. 5A, FIG. 12A-12C). IFNGR1 expression is specifically lost in fibroblast, and not affected in keratinocytes, melanocytes, immune cells or endothelium cells in skin. To investigate whether the IFN-γ responsive dermal fibroblast is the main cell type mediating local CD8+ T cell aggregation and activation, we used the Pdgfra-CreER::IFNGR1 fl/fl cKO mice to induce vitiligo. At Day33 after vitiligo induction, both WT and IFNGR1 cKO mice show robust infiltration of CD8+ T cells into tail skin epidermis (FIG. 5B). But only in WT tail skin CD8+ T cells aggregate into clusters leading to melanocyte loss in the same region; in IFNGR1 cKO tail skin infiltrated CD8+ T cells uniformly distribute throughout the skin without aggregating into clusters and there is no melanocyte loss. We quantified the CD8+ T cell number and remaining melanocytes in each scale of vitiligo induced WT and IFNGR1 cKO tail skin. In WT mice the CD8+ T cell number negatively correlates with melanocyte cell number (R=−0.93, p=2.2×10⁻¹⁶); but in IFNGR1 cKO vitiligo skin, CD8+ T cell number shows no correlation with melanocyte number (R=−0.08, p=0.31) (FIG. 5C). Smoothscatter density plot images show the clone like CD8+ T cell clusters in WT vitiligo skin but completely even distribution in IFNGR1 cKO skin (FIG. 5B). To quantify this phenomenon we analyzed CD8+ T cell cluster size in tail skin of vitiligo induced WT and IFNGR1 cKO mice at Day33 after vitiligo induction. In IFNGR1 cKO vitiligo skin the CD8+ T cell cluster size is significantly smaller than those found in WT vitiligo skin (FIG. 5D). Basically the Pdgfra-CreER::IFNGR1 fl/fl cKO mice phenocopy the IFNGR1 KO mice and show the same defect in CD8+ T cell local aggregation and cytotoxic activity after vitiligo induction. This striking result demonstrates that fibroblast is the main cell type responsible for orchestrating local CD8+ T cell aggregation and activation after being stimulated by IFN-γ in autoimmune skin.

To validate this conclusion and further mechanistically dissect the downstream factors, we used fibroblast mosaic knockdown approach by intradermal injection of lentivirus expressing different shRNAs (FIG. 5E). First we characterized the infected cell types using intradermal injection: lentivirus expressing shRNA and H2BRFP was injected into tail skin dermis of P1 K14H2BGFP mice, then skin sample was collected at P56 for FACS analysis (FIG. 12D). In epidermis, no RFP+ cells are detected. In dermis, RFP+ cells are detected in 3 cell types: CD45−, CD31− stromal fibroblasts, CD45−, CD31+ endothelium cells, and CD45+ immune cells. Quantification shows ˜75% of the RFP+ cells to be fibroblasts. For the knockdown and vitiligo induction experiments, lentivirus expressing shRNA and H2BRFP was injected into tail skin dermis of P1 C57 mice; vitiligo was induced 9 weeks later. At Day33 post induction, T cell infiltration and melanocyte loss in tail skin was analyzed by wholemount immunofluorescent staining. RFP+ cells mark the distribution pattern of shRNA expressing dermal fibroblasts. In the dermis expressing scrambled shRNA, CD8+ T cells distribution patterns randomly overlap with the RFP+ regions; in the corresponding epidermis, CD8+ T cells distribution patterns largely follower their own dermis populations as a mirror image, and wherever there are high density CD8+ T cell clusters melanocytes are lost (FIG. 5E). When we compared the density plot images of dermis RFP+ region and corresponding epidermis melanocyte remaining area, there are no discernible patterns. To quantify these results we compared the number of CD8+ T cell vs. the percentage of infected fibroblasts in each unit area of dermis, as well as the percentage of melanocyte remaining area in each unit area of epidermis vs. the percentage of infected fibroblasts in the corresponding dermis unit (FIG. 5F). In the infection alone but no vitiligo induced mice, there are no infiltrated CD8+ T cells in the dermis, hence no correlation between the number of CD8+ T cell vs. the percentage of infected fibroblasts (R=0.13, p=0.18); since melanocytes are all intact in epidermis there is no correlation between the percentage of melanocyte remaining area vs. the percentage of infected fibroblasts either (R=0.013, p=0.89). This result indicates lentivirus infection in dermis does not trigger CD8+ T cells infiltration in skin or melanocyte loss. In the scrambled shRNA expressing skin with vitiligo induction, there is significant increase of infiltrated CD8+ T cells in the dermis. But the number of CD8+ T cells in the RFP+ high and low regions are equivalent, so there is no correlation between the number of CD8+ T cell vs. the percentage of infected fibroblasts (R=0.04, p=0.33); also even though there are loss of melanocytes in epidermis, the decreases are the same in RFP+ high and low regions, so there is no correlation between the percentage of melanocyte remaining area vs. the percentage of infected fibroblasts either (R=0.05, p=0.29). This result shows mosaic expression of scrambled shRNA in dermal fibroblasts does not affect local CD8+ T cells aggregation and cytotoxic activity after vitiligo induction.

So next we tested that dermal fibroblasts knockdown of key IFN-γ signaling mediators would inhibit CD8+ T cell local aggregation and activation in WT mice. We picked 2 shRNAs against each gene of IFNGR1, JAK1 or STAT1 with high knockdown efficiency, and their effects in blocking IFN-γ induced response were confirmed in cultured fibroblasts in vitro (FIG. 12E-12F. As shown in FIG. 5E, in the dermis expressing JAK1 shRNA, CD8+ T cells gravitate towards the RFP-WT fibroblasts region in the mosaic skin, and are largely absent in the RFP+ JAK1 shRNA expressing fibroblasts region. In the corresponding epidermis, CD8+ T cells distribution patterns reflect their own dermis populations and result in melanocytes loss in the CD8+ T cell high density regions. When we compared the density plot images of dermis RFP+ region and corresponding epidermis melanocyte remaining area, they largely overlap meaning JAK1 shRNA expressing fibroblasts protect the overhead melanocytes of the same region within the mosaic skin. Quantifications show that in skins expressing shRNAs against IFNGR1, JAK1 or STAT1, the numbers of CD8+ T cells in the RFP+ high regions are lower than those in the RFP-regions. Overall the numbers of CD8+ T cell negatively correlate with the percentage of infected fibroblasts: IFNGR1 shRNAs (R=−0.43, p<2.2×10⁻¹⁶); JAK1 shRNAs (R=−0.5, p<2.2×10⁻¹⁶); STAT1 shRNAs (R=−0.47, p<2.2×10⁻¹⁶). Correspondingly, the loss of melanocytes in epidermis is higher in the RFP− regions than in the RFP+ regions. Overall the percentages of melanocyte remaining area positively correlate with the percentage of infected fibroblasts: IFNGR1 shRNAs (R=0.29, p<1.9×10⁻¹⁶); JAK1 shRNAs (R=0.39, p<2.2×10⁻¹⁶); STAT1 shRNAs (R=0.38, p<2.2×10⁻¹⁶).

FIG. 5A shows schematic diagram and QPCR validation of fibroblast specific ablation of IFNGR1 using Pdgfra-CreER::IFNGR1 fl/fl mice. After tamoxifen injection from P50 to P56, FACS purified keratinocytes, melanocytes, immune cells, endothelial cells and fibroblasts were used for QPCR analysis; FIG. 5B shows representative density plot images of CD8+ T cells and whole-mount immunofluorescent staining images of CD8+ T cells and DCT+ melanocytes in tail skin epidermis of vitiligo-induced WT and Pdgfra-CreER::IFNGR1 fl/fl mice. SmoothScatter density plot images indicate the distribution and density of T cells (blue) corresponding to the whole amount immunofluorescent staining images. At Day33 after vitiligo induction in WT and IFNGR1 cKO mice, tail skin epidermis show robust infiltration of CD8+ T cells. But only in WT tail skin, CD8+ T cells aggregate into clusters and lead to melanocytes loss in the same region. In IFNGR1 cKO tail skin CD8+ T cells uniformly distribute without aggregating into clusters and there are no melanocyte loss. Scale bar, 500 um; FIG. 5C shows scatter plots and linear regression lines of CD8+ T cell number vs. percentage of melanocytes in each scale of vitiligo-induced WT and Pdgfra-CreER::IFNGR1 fl/fl mice tail skin. In WT vitiligo skin, CD8+ T cell number negatively correlates with melanocyte cell number (R=−0.93, p=2.2×10⁻¹⁶), but in IFNGR1 cKO vitiligo skin, CD8+ T cell number show no correlation with melanocyte cell number (R=−0.08, p=0.31); FIG. 5D shows size distribution of T cell clusters as a function of vitiligo induction time at Day33 in WT and Pdgfra-CreER::IFNGR1 fl/fl skin; FIG. 5E shows schematic diagram, representative whole-mount immunofluorescent staining images and density plot of dermal knockdown experiments. Lentivirus expressing shRNA and H2BRFP was injected into P1 tail skin dermis. Vitiligo was induced at 9 weeks and whole mount staining was performed 33 days later. RFP marks lentivirus infected dermal fibroblasts. CD8a staining marks infiltrated CD8+ T cells in epidermis or dermis. DCT staining marks melanocytes in epidermis. SmoothScatter density plot images indicate the distribution and density of shRNA expressing fibroblasts (red), T cell (blue), and melanocytes (grey) corresponding to the whole mount immunofluorescent staining images. Scale bar, 500 um; FIG. 5F shows Box-whisker scatter plots and linear regression lines of T cell number vs. percentage of infected fibroblasts (upper panels), and percentage of melanocyte remaining area vs. percentage of infected fibroblasts (lower panels) in each scale of vitiligo-induced tail skin in dermal knockdown experiments. R indicates the correlation, p indicates the significance of the correlation.

FIG. 12A shows schematic diagram and FACS profiles of isolating different cell populations in skin epidermis and dermis. Epidermis and dermis of tail skin were enzymatically dissociated with dispase treatment. Then skin epidermis was digested with trypsin and dermis was digested with collagenase to obtain single cell suspensions. After immunostaining of c-Kit, CD45, or CD31, different cell types were collected using FACS. In epidermis, CD45 and c-Kit were used to enrich immune cells (CD45+, c-Kit−), melanocytes (CD45−, c-Kit+), and keratinocytes (CD45−, c-Kit−). In dermis, CD45 and CD31 were used to distinguish immune cells (CD45+, CD31−), endothelial cells (CD45−, CD31+) from stromal fibroblasts (CD45−, CD31−); FIG. 12B shows QPCR analysis of cell type specific genes in FACS isolated populations indicated in FIG. 12A. KRT14, DCT, CD45, PDGFRA, and CD31 were used as signature genes of keratinocyte, melanocyte, immune cell, fibroblast, and endothelial cell respectively. Note each signature gene was only highly expressed in the intended cell type, indicated the specificity of the cell types isolated using FACS; FIG. 12C shows representative section immunofluorescent staining images of Pdgfra-CreER::mTmG tail skin after tamoxifen injection. K14 staining marks epithelial cells; CD31 staining marks endothelial cells. mGFP signal marks the Pdgfra-CreER labeled cells. The mutually exclusive patterns of K14 and CD31 with mGFP indicate Pdgfra-CreER labels most skin dermal fibroblasts but not kerationcytes and endothelial cells; FIG. 12D shows schematic diagram and FACS profiles of different cell populations in skin epidermis and dermis after intradermal injection of lentivirus. Lentivirus expressing shRNA and H2BRFP was injected into tail skin dermis of P1 K14H2BGFP mice. Skin sample was collected at P56 for FACS analysis. Epidermis and dermis of tail skin were enzymatically dissociated with dispase treatment. Then skin epidermis was digested with trypsin and dermis was digested with collagenase to obtain single cell suspensions. After immunostaining of CD45 and CD31, percentage of different cell types in RFP+ cells were analyzed using FACS. In epidermis, no RFP+ cells were detected in GFP+ keratinocytes. In dermis, RFP+ cells were separated into 3 main cell types present in tail skin dermis: CD45+,CD31− immune cells; CD45−, CD31+ endothelium cells; and CD45−,CD31− stromal fibroblasts. Quantification result shows the cell type composition in RFP+ cells after intradermal injection of lentivirus; FIG. 12E shows schematic diagram and qPCR validation of shRNA knock down efficiency. Two shRNAs targeting each IFN-γ signaling pathway component genes IFNGR1, JAK1 and STAT1 were used. Lentivirus expressing shRNA and H2BRFP were packaged in 293 ft cells and used to infect 3T3 fibroblasts in vitro. Lentivirus expressing scrambled shRNA was used as control. Knockdown efficiency of each shRNA was compared to scrambled shRNA expressing cells; FIG. 12F shows schematic diagram and qPCR analysis of shRNA effect in blocking IFN-γ response. After lentivirus infection, 3T3 cells were treated with IFN-γ for 6 hrs before being collected for qPCR analysis. Classic IFN-γ target gene GBP2 was used as a reporter for activation of IFN-γ signaling pathway. Lentivirus expressing scrambled shRNA was used as control. After 6 hrs IFN-γ treatment, GBP2 expression level significantly increased in cells expressing scrambled shRNA. Knockdown of IFNGR1, JAK1 and STAT1 all significantly reduced IFN-γ induced GBP2 increase. (Scale bar, 50 μm. Data reflect mean±SD from 3 independent mice or experiments with technical triplicates).

The fibroblast mosaic knockdown experiments not only validate the result we obtained using the Pdgfra-CreER::IFNGR1 fl/fl mice, they also further reveal the IFNGR1-JAK1-STAT1 signaling axis in fibroblasts is required for mediating CD8+ T cell local aggregation and activation. Most importantly these experiments show that in a field with uneven fibroblast response to IFN-γ, T cells preferentially aggregate towards regions with high IFNGR1-JAK1-STAT1 signaling.

### Example 6 IFN-γ Signaling Induced Fibroblast-Specific Secreted Chemokines Control Regional T Cell Recruitment

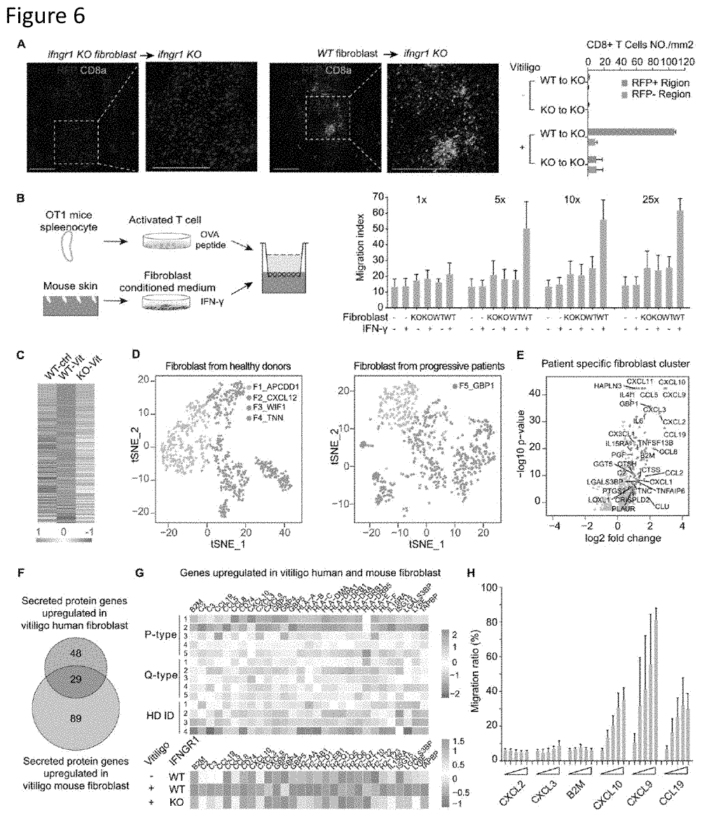

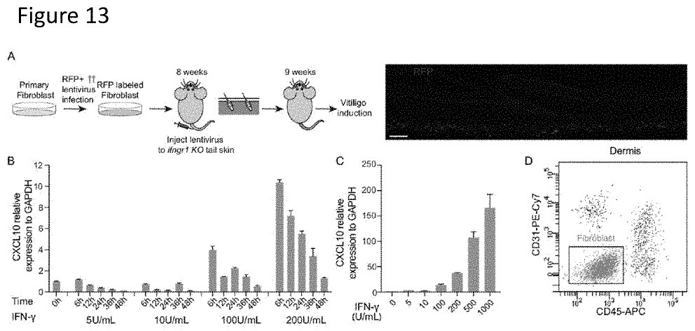

To test if purified fibroblasts alone are sufficient to induced T cell local aggregation in vivo, we isolated primary fibroblasts from WT and IFNGR1 KO mice skin, labeled them with RFP and intradermally injected into IFNGR1 KO mice tail skin followed by vitiligo induction in the host mice (FIG. 13A). CD8+ T cell aggregation was analyzed by whole-mount immunofluorescent staining and quantification. Injection of IFNGR1 KO fibroblasts into the tail skin of IFNGR1 KO mice did not result in local CD8 T cell aggregation after vitiligo induction. But injection of WT fibroblasts alone into the tail skin dermis of IFNGR1 KO mice results in local CD8 T cell aggregation after vitiligo induction. Quantification shows, without vitiligo induction no CD8+ T cells can be detected in the IFNGR1 KO skin with either WT or IFNGR1 KO fibroblasts injections; but after vitiligo induction, only in the RFP+WT fibroblasts region there are increased CD8+ T cells, while in the RFP− region of the same skin there are very little CD8+ T cells, similar to the RFP+/−IFNGR1 KO fibroblasts injected conditions (FIG. 6A). These findings provide clear evidence that in an otherwise completely IFN-γ non-responsive local environment, IFN-γ responsive fibroblasts alone are sufficient to orchestrate local CD8+ T cells aggregation.

To distinguish if this effect is dependent to cell-cell contact or secreted factors, we employed in vitro T cell transwell migration assay with IFN-γ treated fibroblasts conditioned medium (FIG. 6B). Maximum activation effect of IFN-γ treatment dosage and time are optimized by in vitro assays first (FIG. 13B-13D). Then primary fibroblast isolated from WT and IFNGR1 KO skin are treated with 1000 U/mL IFN-γ for 6 hrs in vitro, and the conditioned medium are concentrated to different degree before being added to the lower chamber of the transwell plates. OT1 splenocytes obtained from OT1 mice are activated by OVA peptide in vitro. The activated T cells are added to the upper chamber of the transwell plates. T cell transwell migration index is calculated based on FACS quantification of cell numbers in upper and lower chambers. Of all the medium tested, only the conditioned medium from IFN-γ treated WT fibroblast can induce CD8+ T cell migration in a concentration dependent manner. In contrast, IFN-γ treated IFNGR1 KO fibroblasts conditioned medium has no such chemotaxis effect (FIG. 6B). This result indicates IFN-γ responsive fibroblasts mediate CD8+ T cells aggregation through secreted factors.

To further identify the secreted factors involved in this process, we first performed RNA-seq analysis of fibroblasts from WT and IFNGR1 KO mice at Day 33 after vitiligo induction, alongside WT mice without vitiligo induction (FIG. 6C). Bioinformatics analysis shows among the 432 genes upregulated in WT vitiligo fibroblasts in comparison to both WT control fibroblasts and IFNGR1 KO vitiligo fibroblasts (>2 fold differentially expressed, p<0.01), 118 of them encode secreted protein. Next we compared the single cell transcriptome data of progressive state vitiligo patients fibroblasts and healthy donors fibroblasts. Unsupervised clustering performed by spectral clustering method separates fibroblasts into 4 sub-clusters in healthy donors and 5 sub-clusters in progressive state vitiligo patients (FIG. 6D). In the unique fibroblast cluster present in progressive state vitiligo patients, 59 of the enriched genes (>1.5 fold upregulated, p<0.01) encode secreted protein (FIG. 6E). Comparison of the human and mouse vitiligo fibroblasts specific secreted protein gene signatures, we identified 29 overlapped genes. Among them 11 are conserved major histocompatibility complex genes and 6 are chemokines, including CCL5, CCL8, CCL19, CXCL3, CXCL9 and CXCL10 (FIG. 6F-6G). To functionally test the ability of these secreted chemokines in promoting CD8+ T cells migration, we incubated activated CD8+OT1 T cells in transwells in the presence of various chemokines in lower chamber (FIG. 6H). Among the chemokines tested, CXCL9, CXCL10 and CCL19 induce CD8+ T cell migration in a dosage dependent manner.

FIG. 6A shows representative whole-mount immunofluorescent staining images and quantification of intradermal fibroblasts injection experiments. RFP expressing WT or IFNGR1 KO fibroblasts were intradermally injected into the tail skin of IFNGR1 KO mice. After vitiligo induction on the host mice, CD8+ T cell infiltration was analyzed by whole-mount immunofluorescent staining and quantification. Injection of IFNGR1 KO fibroblasts into the tail skin of IFNGR1 KO mice did not result in local CD8+ T cell aggregation after vitiligo induction. Only injection of WT fibroblasts into the tail skin of IFNGR1 KO mice resulted in local CD8+ T cell aggregation after vitiligo induction. Scale bar, 500 um. Data reflect mean±SD from 3 independent host mice; FIG. 6B shows schematic diagram and quantification of T cell transwell migration assay. Primary skin fibroblasts isolated from WT or IFNGR1 KO mice were treated with 1000 U/mL IFNγ for 6 hrs in vitro. Then the conditioned medium were concentrated to different degrees and added to the lower chambers of the transwell plates. OT1 splenocytes obtained from OT1 mice were activated by OVA peptide in vitro. The activated T cells were added to the upper chambers. Cells were collected from both chambers and counted by flow cytometry after 3 hr incubation. CD8 T cell migration index was calculated by dividing the number of migrated cells (cells in lower chamber) using the number of migrated plus unmigrated cells (cells in upper chamber+lower chamber); FIG. 6C shows heatmap of differentially expressed genes in tail skin fibroblasts from WT control, WT vitiligo-induced and IFNGR1 KO vitiligo-induced mice. Genes were selected based on >2 fold differentially expressed (p<0.01) between WT vitiligo vs. WT control, and WT vitiligo vs. IFNGR1 KO vitiligo; FIG. 6D shows the t-SNE projection of fibroblasts from progressive state vitiligo patients and healthy donors. Unsupervised clustering performed by spectral clustering method separated fibroblasts into 4 sub-clusters in healthy donors and 5 sub-clusters in progressive state vitiligo patients. Each cluster is colored and annotated based on signature gene expression pattern: F1_APCDD1 (red), F2_CXCL12 (green), F3_WIF1 (blue), F4_TNN (purple) and F5_GBP1 (orange). F5 cluster is uniquely present in progressive state vitiligo patients; FIG. 6E shows volcano plot of differentially expressed genes in fibroblasts of progressive state vitiligo patients compared to healthy donors. Red dots in volcano plot denote genes >1.5 fold upregulated (p<0.01) in fibroblasts from progressive state vitiligo patients; FIG. 6F shows venn diagram of genes encoding secreted proteins commonly up-regulated in WT vitiligo-induced mouse fibroblasts and progressive state vitiligo patient specific fibroblasts. RNA-seq analysis of fibroblasts from Day 33 vitiligo mouse skin compared to control mouse skin identified a total of 118 secreted protein genes (fold change>1.5 and p-value <0.01) specifically expressed in WT vitiligo-induced mouse fibroblasts. Single cell RNA-seq analysis identified a total of a total of 77 secreted protein genes (fold change>1.5 and p-value<0.01) specifically expressed in progressive state vitiligo patient fibroblasts compared to healthy donors. Overlap of these two secreted protein gene signatures from mouse and human vitiligo fibroblasts reveals 29 overlapped genes, including 11 conserved major histocompatibility complex genes; FIG. 6G shows heatmap analysis of the 29 commonly up regulated secreted protein genes from (FIG. 6F) in fibroblasts from human or mice at indicated conditions. Upper panel: expression pattern of the 29 genes in fibroblasts from vitiligo patients and healthy donors. Patient ID and disease states are marked alongside. Lower panel: expression pattern of the 29 genes in fibroblasts from WT or IFNGR1 KO mice with or without indicated vitiligo induction. Mouse genotype and vitiligo induction condition are marked alongside; FIG. 6H shows quantification of T cell transwell migration assay. Activated T cells were added to the upper chamber of transwell plates. Recombinant proteins of the indicated cytokines at increasing concentrations were added in the lower chambers. After 3 hr incubation, CD8+ T cell migration index was calculated by dividing the number of migrated cells (cells in lower chamber) using the number of migrated plus unmigrated cells (cells in upper chamber+lower chamber).

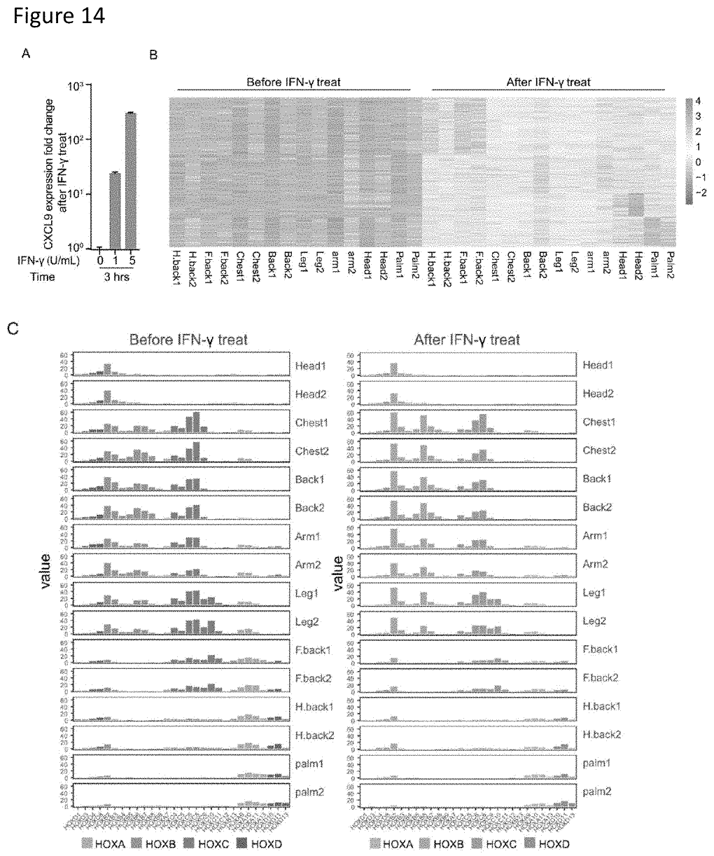

FIG. 13A shows schematic diagram and representative section fluorescent image of fibroblasts intradermal injection experiment. Primary fibroblasts isolated from WT or IFNGR1 KO newborn mice dorsal skin were infected with lentivirus expressing shRNA and RFP in vitro. The RFP+ fibroblasts were then intradermally injected into P56 mice tail skin. The presence and location of injected cells were examined 2 weeks later. Note the injected fibroblasts mainly localized to the lower dermis region in section image; FIG. 13B shows QPCR analysis of IFN-γ treated primary fibroblasts using target gene CXCL10 as the readout. Primary mouse fibroblasts were treated with IFN-γ of increasing dosage and duration time in vitro. Treatment time ranges from 6 hrs to 48 hrs; and IFN-γ concentration gradients range from 5 U/mL to 200 U/mL. CXCL10 expression is induced by IFN-γ in a dosage dependent manner. But prolonged treatment leads to diminishing induction effect; FIG. 13C shows QPCR analysis of CXCL10 gene expression level to optimize IFN-γ treatment dosage for maximum activation effect. Primary mouse fibroblasts were treated with increasing dosage of IFN-γ for 6 hrs in vitro. IFN-γ concentration gradients range from 5 U/mL to 1000 U/mL. Based on the CXCL10 expression level, maximum activation was induced by 1000 U/mL IFN-γ; FIG. 14C shows representative FACS profile of isolating tail skin dermal fibroblasts used for RNA-seq analysis. Epidermis and dermis of tail skin were enzymatically dissociated with dispase treatment. Then skin dermis was digested with collagenase to obtain single cell suspensions. After immunostaining of CD45 and CD31, fibroblasts were collected using FACS as the CD45−, CD31− population. (Scale bar, 100 μm. Data reflect mean±SD from 3 independent experiments with technical triplicates)

Taken together, IFN-γ responsive fibroblasts are sufficient to mediate CD8+ T cells aggregation in vivo and in vitro through secreted chemokines such as CXCL9, CXCL10 and CCL19.

### Example 7 Intrinsic IFN-γ Response Differences of Anatomically Distinct Human Fibroblasts Correlate with Regional Disease Variations

Our findings here revealed that skin dermal fibroblasts are necessary and sufficient to induce CD8+ T cell local aggregation and activation in response to IFN-γ in vitiligo skin. One of the most intriguing features of vitiligo is the commonly occurring bilateral symmetric depigmentation pattern in majority of the non-segmental vitiligo patients. We tried to confirm some local cue is involved in recruiting immune cells into bilateral symmetric body positions.

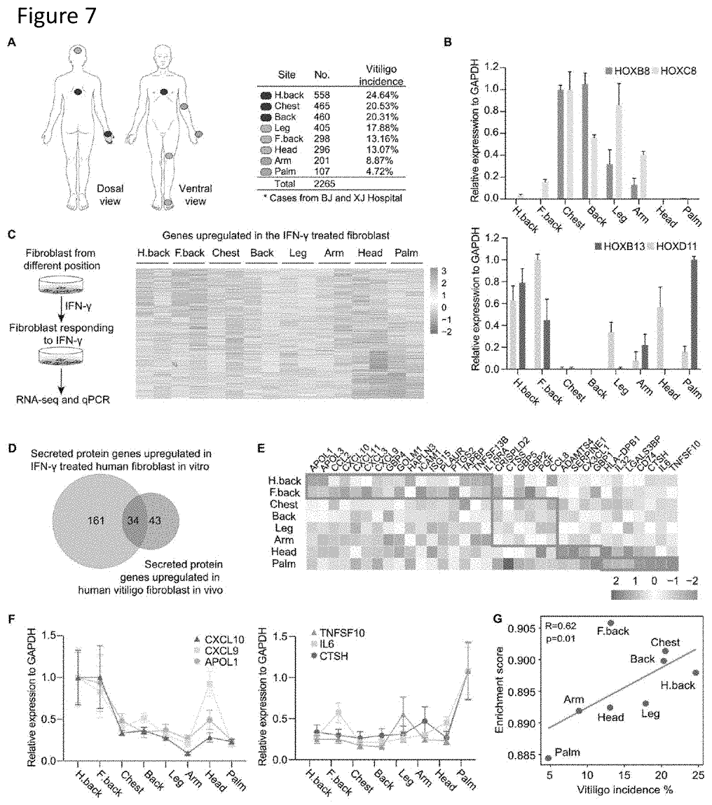

We first quantified vitiligo incidence frequencies at different body positions. Although any part of skin in the body could be affected by vitiligo, clinical evidence suggested depigmentation usually appeared in several specific body regions. We analyzed the lesion position of 2265 non-segmental vitiligo patients from Beijing Hospital and Xijing Hospital in China. The lesion sites are divided into eight main body regions, including hand back, chest, back, leg, food back, head, arm, and palm. The vitiligo incidence frequency at each position is calculated by dividing the number of patients with lesional skin depigmentation at the indicated body region with the total patient numbers. Result shows large variations of vitiligo incidence in the eight body regions: with hand back, chest and back skin regions to be the most susceptible to vitiligo, while palm and arm skin to be the least susceptible (FIG. 7A)

Next, we sought to determine whether anatomically distinct fibroblast have intrinsic differences in IFN-γ signaling response by conducting RNA-seq analysis of primary human skin dermal fibroblasts from 8 body positions with or without IFN-γ treatment in vitro (FIG. 14, FIG. 7C). HOX gene expression patterns indicate the fibroblasts retain their positional information during in vitro culture (FIG. 7B). We identified 1195 genes that are upregulated in fibroblasts of at least one body position after IFN-γ treat (FIG. 7C). Heatmap analysis of the combined upregulated genes among the 8 body positions reveals that the up-regulated genes varied from different body positions. Hand back and foot back fibroblasts exhibit similar IFN-γ response transcriptional signatures. Chest, back, leg and arm share similar upregulated genes. Head and palm respectively show distinct transcriptional profiles from the other body positions (FIG. 7C). These results suggest that fibroblasts from anatomically distinct body positions show intrinsic differences in IFN-γ response. From the combined upregulated gene list, we identified a total of 195 secreted protein genes enriched in the IFN-γ activated fibroblasts and 34 of them overlap with those identified in progressive vitiligo patients fibroblasts, including several chemokine genes (FIG. 7D). The chemokine genes CCL2, CXCL3, CXCL9, CXCL10, and CXCL11 are mainly upregulated in the skin fibroblasts from hand back and foot back (FIG. 7E). QPCR validations confirmed the intrinsic IFN-γ response differences of fibroblasts from different anatomic positions (FIG. 7F). Finally to test whether or not the intrinsic IFN-γ response difference of regional fibroblasts could explain the regional disease incidence difference, we evaluated the correlation of IFN-γ response enrichment score to vitiligo incidence at different body positions (FIG. 7G). Result shows vitiligo incidence positive correlates with the Intrinsic IFN-γ response of skin fibroblast (R=0.62, p=0.01).

FIG. 7A shows quantification of lesion frequencies at anatomically distinct skin regions in vitiligo patients. The vitiligo incidence at each position is calculated as the number of patients with lesion site at this body region divided by the total patients number, which is 2265 non-segmental vitiligo patients documented in Beijing Hospital and Xijing Hospital. The 8 anatomic positions include palm, hand back, arm, chest, back, leg, foot back, and head; FIG. 7B shows QPCR analysis of HOXB8, HOXC8, HOXB13 and HOXD11 in fibroblasts from 8 anatomic positions. Result shows HOXB8 and HOXC8 are enriched in fibroblasts from chest, back, leg, and arm while the HOXB13 and HOXD11 are enriched in fibroblasts from hand back, foot back, head, and palm. Data reflect mean±SD from 3 independent individuals with technical triplicates; FIG. 7C shows schematic diagram and heatmap analysis of in vitro IFN-γ treated fibroblasts isolated from 8 anatomically distinct positions. RNA-seq analysis of fibroblasts treated with 1 U/mL recombinant IFN-γ identified responsive genes in fibroblasts from each anatomic positions (fold change>2 and p-value <0.01). Columns show fibroblasts from 8 body positions of 2 individuals; rows show differential expressed genes that are ordered by the p-value. A total of 1167 differential expressed genes were displayed. Data is normalized for each gene using the Scale function of R; FIG. 7D shows venn diagram depicts that 34 secreted protein genes commonly up-regulated in IFN-γ treated fibroblasts in vitro and fibroblasts from progressive state vitiligo patients in vivo. A total of 195 secreted protein genes are up-regualted in the IFN-γ treated fibroblasts and 34 of them overlapped with secreted protein genes up-regualted in fibroblasts from progressive state vitiligo patients; FIG. 7E shows heatmap analysis of the 34 commonly up-regulated secreted protein genes in IFN-γ treated fibroblasts from 8 anatomically distinct positions. Skin region are marked alongside. Boxes highlight genes specifically up-regulated in fibroblasts from certain anatomic positions. Data was calculated by averaging the values from 2 individuals, and was normalized for each gene using the Scale function of R; FIG. 7F shows QPCR analysis of vitiligo fibroblast signature genes in fibroblasts from 8 anatomically distinct positions after IFN-γ treatment. Data reflect mean±SD from 3 independent individuals with a technical triplicate; FIG. 7G shows scatter plot and linear regression line shows the correlation of intrinsic IFN-γ response differences of skin fibroblast vs. vitiligo incidences at 8 anatomically distinct positions. Intrinsic IFN-γ response differences are reflected by the enrichment score, which is calculated by the Gene Set Enrichment Analysis (GSEA) with the 1167 up-regulated genes from distinct regional fibroblast after IFN-γ activation. Result shows vitiligo incidence positive correlates with the Intrinsic IFN-γ response of skin fibroblast (R=0.62, p=0.01).

FIG. 14A shows QPCR analysis of IFN-γ target gene CXCL9 in the primary human fibroblast after indicated IFN-γ treatments. Primary human fibroblasts were treated with IFN-γ of increasing dosage and duration time in vitro. IFN-γ concentration gradients range from 0 to 5 U/mL. Result shows treatment of IFN-γ at 1 U/mL for 3 hrs is within the logarithmic activation range. Data reflect mean±SD from 3 independent experiments with technical triplicates; FIG. 14B shows heatmap of upregulated genes in human fibroblast from different body positions after IFN-γ treatment (log 2 fold change >1, and p-val <0.01). Fibroblasts from 8 body positions of two individuals with or without IFN-γ treatment were used for RNA-seq analysis. Columns show 8 body positions from 2 individuals, rows show differential expressed genes. 1195 differential expressed genes were displayed. Gene expression values are normalized for each gene by subtracting the average value of all samples from each sample value. Result shows fibroblasts from each position have a specific series of IFN-γ response signature genes; FIG. 14C shows RNA-seq results of HOX genes expression pattern in fibroblast from 8 different body positions with or without IFN-γ treatment. Rows show 8 body positions from two individuals, columns show expression level of different HOX genes. Each column were colored based on the HOX genes cluster.

Result shows anatomically distinct fibroblasts have specific series of HOX signature genes and the expression patterns were not altered after IFN-γ treatment.

1. Use of IFN-γ signaling as a drug target for vitiligo.

2. The use of claim 1, wherein the drug target is the IFN-γ signaling within a cell in skin.

3. The use of claim 1, wherein the cell in skin is selected from the endothelial cell, the dermal cell, the smooth muscle cell and the immune cell in skin, preferably, the drug target is IFN-γ signaling within the dermal fibroblast, more preferably, the drug target is fibroblast-specific secreting chemokines induced by IFN-γ signaling, more preferably the drug target is IFNGR1-JAK1-STAT1 signaling within dermal fibroblast of skin.

4. The use of claim 3, wherein the chemokine is selected from CCL5, CCL8, CCL19, CXCL3, CXCL9 and CXCL10.

5. (canceled)

6. A method for establishing a vitiligo animal model, or inducing vitiligo in an animal, comprising inoculating the animal with melanoma cells to produce a tumor, injecting CD4 depletion antibody and removing the melanoma before expanding and metastasizing.

7. The method of claim 6, wherein the melanoma cell is a B16F10 melanoma cell.

8. (canceled)

9. The method of claim 6, wherein the animal is mouse, rat, canine, pig or cat.

10. The method of claim 9, wherein the tumor of the mouse is surgically removed after the volume of the melanoma reaching 62.5-256 mm³.

11. A vitiligo animal model, which is obtained using the method of claim 6.

12. The vitiligo animal model of claim 11, wherein the vitiligo animal model has overexpressed IFNGR1-JAK1-STAT1.

13. A skin of an animal obtained using the method of claim 6, having reduced melanocytes.

14. The skin of claim 13, wherein the skin shows depigmentation.

15. The skin of claim 13, wherein the skin is selected from tail skin, back skin, hand back skin, foot back skin, chest skin, leg skin or arm skin of an animal.

16. The skin of claim 13, wherein the animal is mouse, rat, canine, pig or cat.

17. (canceled)

18. An isolated cell of skin of claim 13, wherein the cell is an IFN-γ responsive cell, and is selected from keratinocyte, melanocyte, fibroblast, endothelium cell, smooth muscle cell, and dendritic cell, preferably, the cell is fibroblast, more preferably, the cell is dermal fibroblast, and more preferably, the cell is dermal fibroblast of mouse tail skin.

19. (canceled)

20. The isolated cell of claim 18, wherein the cell is a fibroblast having IFNGR1-JAK1-STAT1 signaling.

21. The isolated cell of claim 18, wherein the cell is an IFN-γ response up-regulated fibroblast from dermis.

22. A method for screening candidate drugs for treating vitiligo using the animal model of claim 11.

23. A method for evaluating the therapeutic effects of vitiligo using the animal model of claim 11.

24. A method for prognosis evaluation of vitiligo using the animal model of claim 11.

25. (canceled)

26. A method for distinguishing disease states of vitiligo using single cell RNA-seq analysis, wherein progressive state vitiligo skin contains more CD8+ cytotoxic T cells that express significant amount of IFNG compared to that in quiescent state patients or healthy donors.

27. (canceled)

28. The method of claim 26, wherein the density of CD8+ T cells positively correlates with the density of pSTAT1+ cells, and the spatial co-distribution pattern of CD8+ T cells and IFN-γ responsive cells in patient skin is consistent with the single cell RNA-seq analysis.
